(19) United States
(12) Patent Application Publication
(10) Pub. No.: US 2014/0290668 A1
(43) Pub. Date: Oct. 2, 2014

(54) Adjustable Breathing Device

(71) Applicants: W. Keith Thornton, Dallas, TX (US); Alastair McAuley, Dallas, TX (US)

(72) Inventors: W. Keith Thornton, Dallas, TX (US); Alastair McAuley, Dallas, TX (US)

(21) Appl. No.: 14/226,968

(22) Filed: Mar. 27, 2014

Related U.S. Application Data
(60) Provisional application No. 61/806,037, filed on Mar. 28, 2013.

(51) Int. Cl. A61M 16/06; A61F 5/56

(52) U.S. Cl. CPC A61M 16/0605; A61F 5/566; A61M 2016/0661; A61M 2207/10; A61M 2207/00; A61M 2202/02
USPC 128/848; 128/205.25; 433/213

(57) ABSTRACT

According to one embodiment, a breathing device includes an oral appliance arch, a post, and first and second masks that are separately adjustable relative to the oral appliance arch. The first mask defines a first interior space and is configured to deliver gas to a user's nose when the first mask is connected to a gas delivery device. The second mask defines a second interior space and is configured to be adjustably secured to the post and to form a substantial seal around the user's oral cavity when pressed against a portion of the user's mouth. The post is coupled to the oral appliance arch and is configured to adjustably secure the first mask relative to the oral appliance arch.

### CROSS REFERENCE TO RELATED APPLICATIONS

This application claims the benefit of U.S. Provisional Application No. 61/806,037 filed Mar. 28, 2013, which is incorporated herein by reference.

### TECHNICAL FIELD

This invention relates generally to medical and dental devices; and more particularly to an adjustable breathing device.

### BACKGROUND

Many people experience breathing problems on a recurring basis, which may result in sleep disordered breathing (i.e., difficulty sleeping, snoring, or other more serious conditions such as obstructive sleep apnea). As technology advances, people with such breathing problems demand increased performance and comfort. Previous devices for improving a user's breathing have included face masks, nose masks, or nasal inserts that help deliver air to the user's nose at positive pressure. Such devices help force open the user's breathing passage and thereby improve the user's breathing. However, such masks may move while the user is sleeping, or they may be uncomfortable to the user when worn.

Other treatments for sleep breathing disorders involve the use of dental devices for extending forward the lower jaw of the patient, which may also operate to more fully open the breathing passage. These dental devices may be created in labs based on a patient's dental impressions. This procedure can cost the patient substantial time and money because the dentist creates a dental impression and then the lab creates the dental device after the dentist sends in the dental impression.

### BRIEF DESCRIPTION OF THE DRAWINGS

FIG. 1 illustrates an example adjustable breathing device.

FIG. 2A illustrates an isometric view of example components that may be used in certain embodiments of an adjustable breathing device.

FIG. 2B illustrates a front view of example components that may be used in certain embodiments of an adjustable breathing device.

FIG. 2C illustrates a side view of example components that may be used in certain embodiments of an adjustable breathing device.

FIG. 3A illustrates an isometric view of example components that may be used in certain alternative embodiments of an adjustable breathing device.

FIG. 3B illustrates a front view of example components that may be used in certain alternative embodiments of an adjustable breathing device.

FIG. 3C illustrates a side view of example components that may be used in certain alternative embodiments of an adjustable breathing device.

FIG. 4A illustrates an isometric view of an example post and arch.

FIG. 4B illustrates a top view of an example post and arch.

FIG. 4C illustrates a front view of an example post and arch.

FIG. 4D illustrates a side view of an example post and arch.

FIG. 5A illustrates an isometric view of an example nose mask.

FIG. 5B illustrates a front view of an example nose mask.

FIG. 5C illustrates a side view of an example nose mask.

FIG. 5D illustrates a side view of a portion of an example nose mask.

FIG. 5E illustrates a front view of a portion an example nose mask.

FIG. 6A illustrates a side view of a portion of an example post.

FIG. 6B illustrates a front view of a portion of an example post.

FIG. 7A illustrates a side view of an example configuration of an example nose mask and post.

FIG. 7B illustrates a side view of an example configuration of an example nose mask and post.

FIG. 7C illustrates a side view of an example configuration of an example nose mask and post.

FIG. 7D illustrates a side view of an example configuration of an example nose mask and post.

FIG. 7E illustrates a side view of an example configuration of an example nose mask and post.

FIG. 7F illustrates a side view of an example configuration of an example nose mask and post.

FIG. 8 illustrates an isometric view of an example post and arched frame.

FIG. 9A illustrates a top view of an example arched frame.

FIG. 9B illustrates a top view of an example arched frame.

FIG. 9C illustrates a side view of an example arched frame.

FIG. 9D illustrates a side view of an example arched frame.

FIG. 9E illustrates an isometric view of an example arched frame.

FIG. 10A illustrates a top view of an example moldable tray.

FIG. 10B illustrates a top view of an example moldable tray.

FIG. 10C illustrates a side view of an example moldable tray.

FIG. 10D illustrates an isometric view of an example moldable tray.

FIG. 11A illustrates a top view of an example arched frame and moldable tray.

FIG. 11B illustrates a side view of an example arched frame and moldable tray.

FIG. 11C illustrates a front view of an example arched frame and moldable tray.

FIG. 12A illustrates an isometric view of an example mouth mask.

FIG. 12B illustrates a side view of an example mouth mask.

FIG. 12C illustrates a front view of an example mouth mask.

FIG. 13A illustrates an isometric view of an example mouth mask and connector.

FIG. 13B illustrates a front view of an example mouth mask and connector.

FIG. 14A illustrates an isometric view of an example nose mask, mouth mask, and connector.

FIG. 14B illustrates a front view of an example nose mask, mouth mask, and connector.

FIG. 14C illustrates a side view of an example nose mask, mouth mask, and connector.

FIG. 15 illustrates an isometric view of an example mouth mask and post.

FIG. 16A illustrates an isometric view of an example tension element.

FIG. 16B illustrates a side view of an example tension element.

FIG. 17A illustrates an isometric view of an example arch and tension element.

FIG. 17B illustrates an isometric view of a portion of an example post.

FIG. 17C illustrates an isometric view of an example arched frame and tension element.

FIG. 17D illustrates a side view of an example nose mask, arches, and tension element.

FIG. 18A illustrates a front view of an example valve.

FIG. 18B illustrates a side view of an example valve.

FIG. 18C illustrates a front view of an example nose mask.

FIG. 19 illustrates an example method of forming a moldable tray at least partially covered by a casing in accordance with certain embodiments of the present disclosure.

FIG. 20A illustrates example casing material and an example moldable tray.

FIG. 20B illustrates example casing material blown down against an example moldable tray.

FIG. 20C illustrates an example moldable tray covered by an example casing.

FIG. 21 illustrates an example moldable tray with an example casing.

FIG. 22 illustrates a side view of an example breathing device using an example separator.

FIG. 23 illustrates an isometric view of an example arch, tension element, and separator.

### SUMMARY OF THE DISCLOSURE

In accordance with the present invention, certain disadvantages and problems associated with an adjustable breathing device may be reduced or eliminated.

According to one embodiment, a breathing device includes an oral appliance arch, a post, and first and second masks that are separately adjustable relative to the oral appliance arch. The first mask defines a first interior space and is configured to deliver gas to a user's nose when the first mask is connected to a gas delivery device. The second mask defines a second interior space and is configured to form a substantial seal around the user's oral cavity when pressed against a portion of the user's mouth. The post is coupled to the oral appliance arch and is configured to adjustably secure the first mask relative to the oral appliance arch. The second mask is configured to be adjustably secured to the post, and the first and second masks are separately adjustable relative to the oral appliance arch.

According to another embodiment, a kit for constructing an adjustable breathing device includes first and second masks, a post configured to adjustably secure the first mask relative to the post, and a plurality of nasal pillows. The first mask defines a first interior space and is configured to deliver gas to a user's nose when the first mask is connected to a gas delivery device. The second mask defines a second interior space and is configured to form a substantial seal around the user's oral cavity when pressed against a portion of the user's mouth. The first and second masks are separately adjustable relative to the post. Each nasal pillow includes first and second ends. The first end defines a circumference and is configured to be positioned proximate to the user's nostril. The second end is configured to couple to the first mask.

According to another embodiment, a breathing device includes first and second masks, a first flange connected to the first mask, an oral appliance arch, and a post coupled to the oral appliance arch. The first mask is configured to deliver gas to a user's nose when the first mask is connected to a gas delivery device. The second mask is configured to form a substantial seal around the user's oral cavity when pressed against a portion of the user's mouth. The post is configured to adjustably secure the first mask relative to the oral appliance arch. The post includes a second flange and a fastener configured to adjustably secure the first flange against the second flange. The second mask is configured to be adjustably secured to the post and to be adjustable in the anterior-posterior direction along the post. The first and second masks are separately adjustable relative to the oral appliance arch.

According to another embodiment a method of forming a breathing device includes [TBD UPON APPROVAL OF CLAIMS]

According to another embodiment, a breathing device includes

According to another embodiment, a kit for constructing an adjustable breathing device includes

Certain embodiments of the invention may provide one or more technical advantages. Example technical advantage of some embodiments may include providing improved comfort, customization, and/or configurability of breathing devices. Certain embodiments may provide separately adjustable nose and mouth masks that may allow the breathing device to be adjusted to fit a variety facial shapes and sizes. Certain embodiments may also provide differently sized and shaped nasal pillows that may accommodate different nostril sizes and positions. Another technical advantage of certain embodiments may include providing user-friendly adjustment mechanisms for adjusting portions of the breathing device. Certain embodiments may also provide more comfortable mechanisms for securing the breathing device against the user's face. Certain embodiments may allow the breathing device to be secured relative to the user's face without straps or other potentially cumbersome implements. Certain embodiments may provide improved securing of the breathing device against the user's face to reduce the risk of the device becoming displaced when the user moves while sleeping. Certain embodiments may also provide improved flexibility and/or durability of certain components of the breathing device. Certain embodiments may provide a breathing device that may couple to an oral appliance to provide an improved fit based on the precise custom orientation or positional relationship between a user's unique facial structure and the user's teeth. Certain embodiments may enable adjustment of the position of the user's lower jaw without locking the user's lower jaw in place, which may provide improved comfort. Certain embodiments may provide a custom-molded arch, which may provide improved comfort and/or fit. Certain embodiments may also provide a simplified process for forming a custom-molded arch, which may reduce the cost and/or inconvenience to the user. Certain embodiments may provide a custom-molded arch with sufficient rigidity to be securely coupled to various attachments while also having sufficient flexibility to be used in mouths of various shapes and/or sizes. Certain embodiments may provide simplified or more cost-effective techniques for forming breathing devices. Furthermore, certain embodiments may provide improved durability, which may mitigate the need for subsequent repair or replacement. Certain embodiments may also provide simplified or more cost-effective mechanism for separating a user's dental arches to increase the area of the oral cavity.

Certain embodiments of the invention may include none, some, or all of the above technical advantages. One or more other technical advantages may be readily apparent to one skilled in the art from the figures, descriptions, and claims included herein.

### DETAILED DESCRIPTION

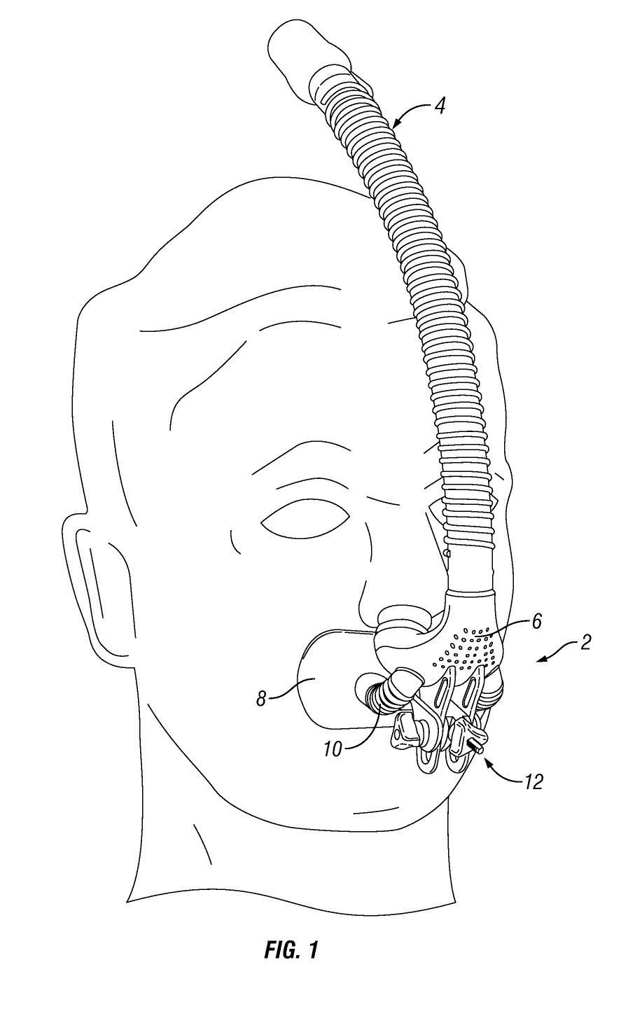

FIG. 1 illustrates an example adjustable breathing device 2. Adjustable breathing device 2 includes tube 4, nose mask 6, mouth mask 8, connector 10, and post 12. Tube 4 may connect to a gas delivery device (not shown) to supply gas to an interior space of nose mask 6. The interior space of nose mask 6 may be connected to the interior space of mouth mask 8 by connector 10. Adjustable breathing device 2 may therefore deliver gas to a user's nose and/or mouth via nose mask 6 and mouth mask 8, respectively. Nose mask 6 and mouth mask 8 may be connected to post 12, which is connected to one or more arches 14 (not shown) that are configured to be positioned proximate to the user's dentition. Nose mask 6 and mouth mask 8 are separately adjustable relative to the user, which may allow adjustable breathing device 2 to accommodate a wide variety of shapes and sizes of faces.

Tube 4 may include any component operable to direct a flow of gas to adjustable breathing device 2 from a gas delivery device. The gas delivery device may provide gas to the user via tube 4 to assist breathing. In some embodiments, gas delivery device may be connected to nose mask 6, mouth mask 8, or to both nose mask 6 and mouth mask 8. Certain embodiments may be used to deliver, for example, pressurized air, oxygen, aerosols, or medication. Particular embodiments may be used for continuous positive airway pressure treatment (CPAP), auto-CPAP, or bilevel or closed-loop ventilation. Tube 4 may be part of nose mask 6, part of the gas delivery device, or a separate component altogether.

Nose mask 6 is configured to deliver gas to a user's nose. For example, nose mask 6 may utilize nasal pillows, which seal against the user's nostrils. As another example, nose mask 6 may seal around the user's nose. Nose mask 6 may be flexible, rigid, or it may include both flexible and rigid portions. Certain embodiments may include nasal pillows 20 (not shown), which extend into the user's nostrils. Nose mask 6 may be adjustably connected to the post 12. Nose mask 6 may be adjusted relative to the user's nose by adjusting its orientation relative to post 12. Providing an adjustable nose mask 6 may allow adjustable breathing device 2 to fit a wide variety of facial shapes and sizes.

Mouth mask 8 is configured to deliver gas to a user's oral cavity. Mouth mask 8 may be flexible, rigid, or it may include both flexible and rigid portions. For example, portions of mouth mask 8 may be made of a flexible material, such as silicone. Mouth mask 8 may include an outer sealing portion configured to foiin a substantial seal around the user's oral cavity. For example, in some embodiments, mouth mask 8 may be configured to form a seal by pressing against the outer surface of the user's face around the mouth. In other embodiments, mouth mask 8 may be configured to fit inside the user's lips while still maintaining a substantial seal to prevent gas from passing around the edges of mouth mask 8. Some embodiments may include a sealing portion configured to press against the outer surface of the user's face around the mouth and a sealing portion configured to fit inside the user's lips. In some embodiments, different types of outer sealing portions may be used interchangeably. The outer sealing portion may also be self-sealing such that the application of positive air pressure to the interior space of mouth mask 8 causes the outer sealing portion to expand to form a seal or improve an existing seal around the oral cavity.

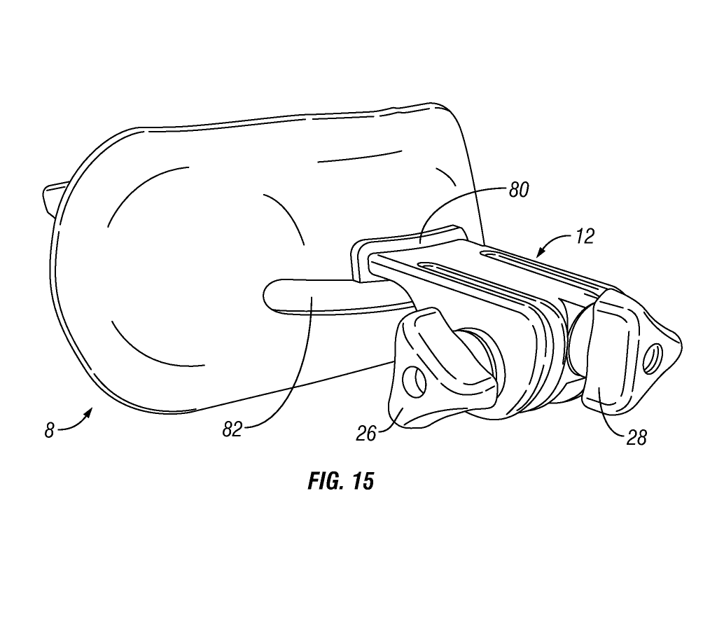

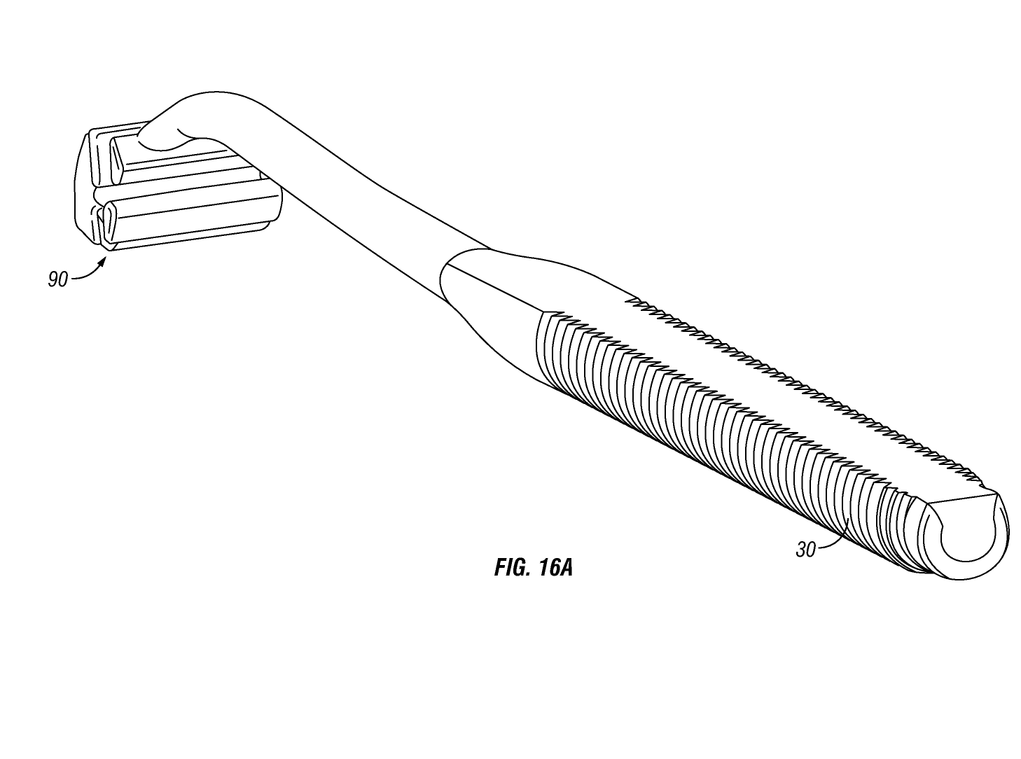

Mouth mask 8 may be adjustable to fit a wide variety of facial shapes and sizes. For example, mouth mask 8 may be adjustably secured along a longitudinal axis of post 12. In some embodiments, mouth mask 8 may include a sealing portion that defines an opening configured to receive post 12, as shown, for example, in FIG. 15. In such embodiments, mouth mask 8 may be substantially secured relative to post 12 by frictional force between the sealing portion of mouth mask 8 and post 12. Additionally or alternatively, mouth mask 8 may be secured against post 12 by a clamp, a set screw, or any other suitable fastener. Providing adjustments in this manner may allow mouth mask 8 to be adjustable to the shape of the user's face while still allowing mouth mask 8 to be anchored relative to the user's dentition, which may provide improved comfort and stability. Mouth mask 8 may also include a portion configured to be positioned under a user's chin, which may help prevent the opening of the user's jaw.

Connector 10 may be any suitable component operable to connect the interior spaces of nose mask 6 and mouth mask 8. For example, connector 10 may include one or more flexible tubes or any other suitable structure. Connector 10 may allow gas delivered to nose mask 6 to pass through to mouth mask 8, which may allow the gas delivery device to deliver gas to the user's nose and mouth without requiring separate connections. When connector 10 is connected to nose mask 6 and mouth mask 8, the interior space of connector 10 may be contiguous with the interior spaces of nose mask 6 and mouth mask 8 such that gas can move from one mask to the other through connector 10. In some embodiments, connector 10 may be removable or absent altogether. Nose mask 6 and/or mouth mask 8 may include one or more plugs to fill the opening created by the absence of connectors 10. In embodiments where mouth mask 8 is not connected to nose mask 6 by connectors 10, mouth mask 8 may operate to seal the user's mouth to prevent gas delivered by nose mask 6 from escaping through the user's mouth. In embodiments where connector 10 is flexible, the flexibility of connector 10 may enable nose mask 6 and mouth mask 8 to be adjusted separately without straining or breaking the connection between them.

Post 12 may be any suitable structure operable to secure adjustable breathing device 2 relative to the user's face. Post 12 may be formed from plastic (e.g., polycarbonate), metal, any other suitable material, or any suitable combination thereof. Post 12 may engage with one or more arches 14 (not shown) positioned inside the user's mouth, and post 12 may extend anterior to the user's mouth when connected to the arches 14. Post 12 may be integrally formed with one or more arches 14 or may be removably coupled to one or more arches 14 (e.g., by snapping, hooking, threading, or otherwise coupling with arch 14). Post 12 may also couple to nose mask 6 and mouth mask 8. For example, post 12 may include one or more couplers operable to adjustably secure nose mask 6 and mouth mask 8 relative to post 12. In some embodiments, post 12 may substantially secure mouth mask 8 via frictional force between a sealing portion of mouth mask 8 and post 12. Additionally or alternatively, mouth mask 8 may be secured against post 12 by a clamp, a set screw, or any other suitable fastener. In certain embodiments, post 12 may include a plurality of ridges or other features to provide distinct adjustment positions for mouth mask 8 along the length of post 12. By engaging arches 14 as well as nose mask 6 and mouth mask 8, post 12 may allow nose mask 6 and mouth mask 8 to be adjustably secured relative to the user's dentition (and therefore relative to the user's nose and mouth).

In certain embodiments, post 12 may be formed and/or molded as one part. In other embodiments, post 12 may be formed from multiple parts that may be assembled, clipped, screwed, or overmolded. In various embodiments, post 12 may include plastic (e.g., polycarbonate), metal (e.g., stainless steel), any other suitable material, or any combination thereof. Post 12 may be sufficiently rigid to prevent substantial deformation outside of the sagittal plane that might hinder the ability of nose mask 6 or mouth mask 8 to deliver gas to the user. Post 12 may also be sufficiently flexible and resilient to withstand forces exerted against post 12 during use (such as, for example, when the user turns during sleep).

Having adjustable breathing device 2 secured to the user's dentition may obviate the need for the user to wear stabilizing straps. Such straps may shift accidentally, may be uncomfortable for the user, may leave marks on the user's face, and may irritate the user's face and scalp. Furthermore, since anchoring nose mask 6 and mouth mask 8 to the user's arches 14 may prevent substantial movement of the masks relative to the user's face and prevent leakage, these embodiments may reduce the need to tighten the masks to the user's face, which may result in reduced pressure on the user's face and reduced pressure sores. These embodiments may also provide greater mask stability during sleep for users who exhibit substantial movement during sleep.

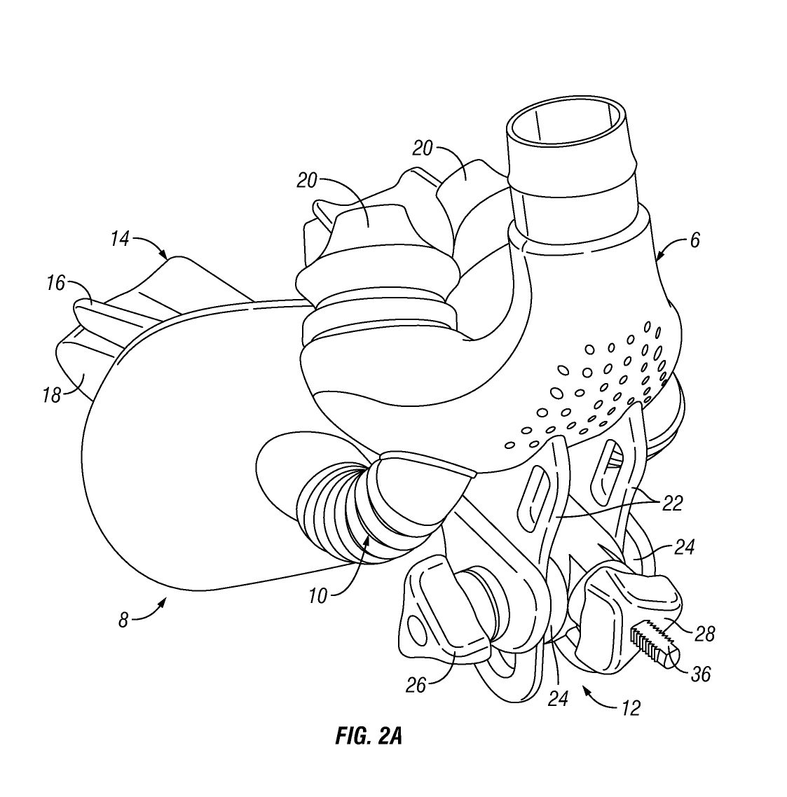

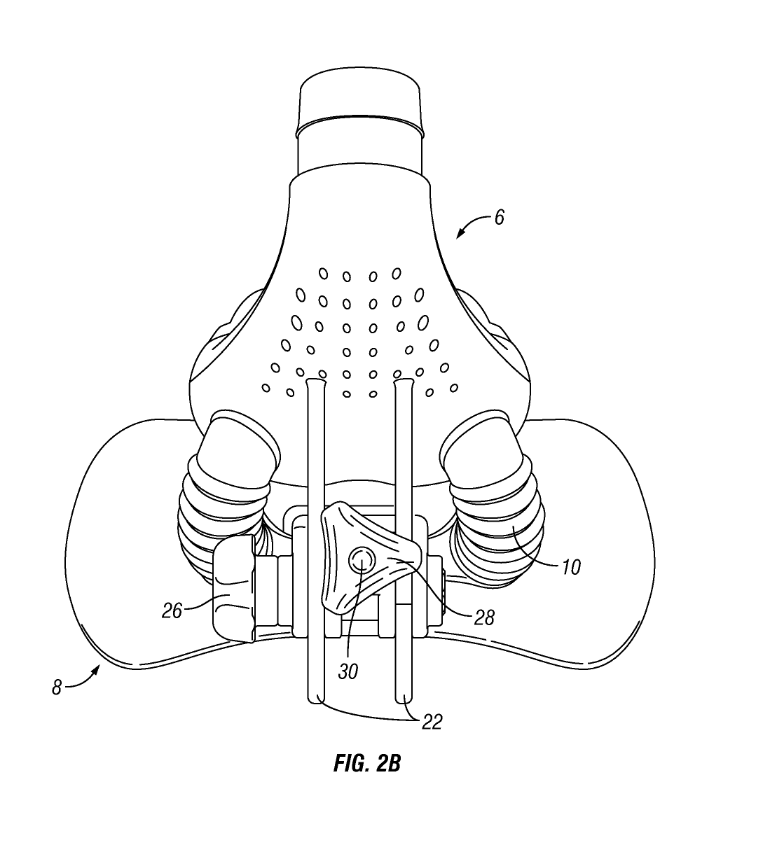

FIGS. 2A-2C illustrate views of example components that may be used in certain embodiments of an adjustable breathing device. FIGS. 2A, 2B, and 2C depict isometric, front, and side views, respectively. Nose mask 6 is connected to mouth mask 8 via connector 10. Nose mask 6 includes flanges 22, which engage with flanges 24 to adjustably secure nose mask 6 against post 12. Post 12 includes flanges 24, tightening knob 26, tension element adjustor 28, and tension element 30.

Nose mask 6, mouth mask 8, connector 10, and post 12 may include any structure, configuration, or function discussed above regarding FIG. 1.

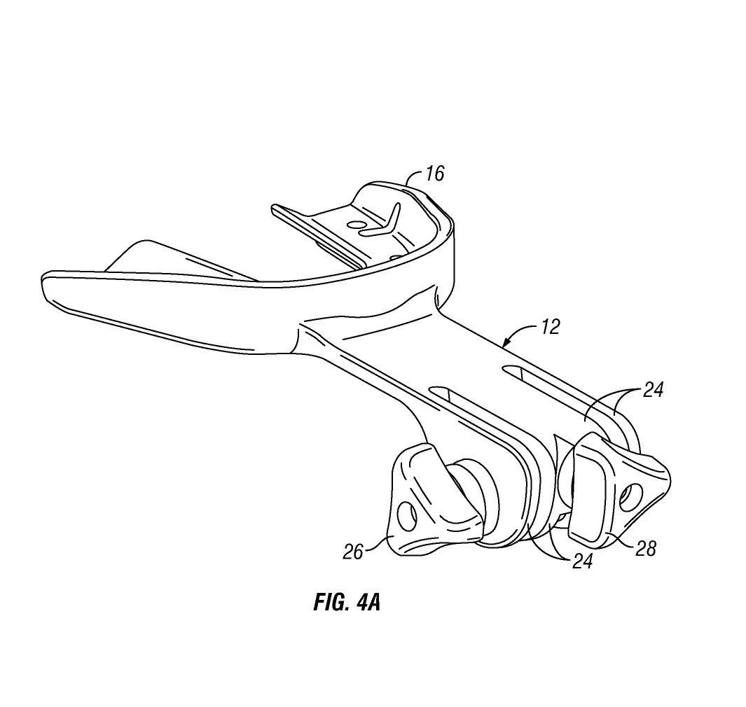

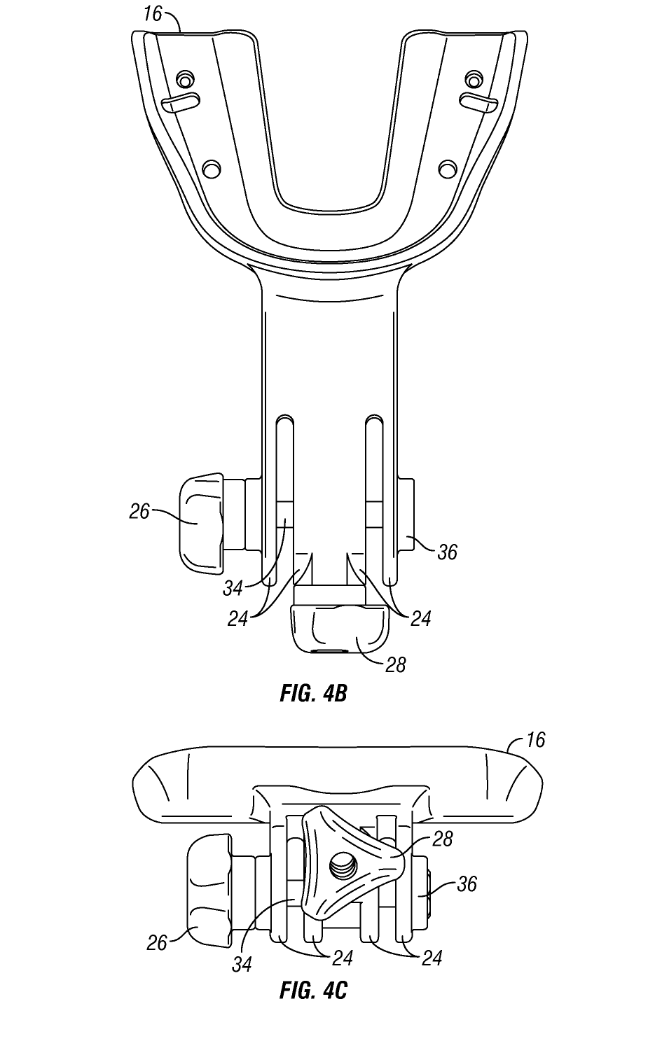

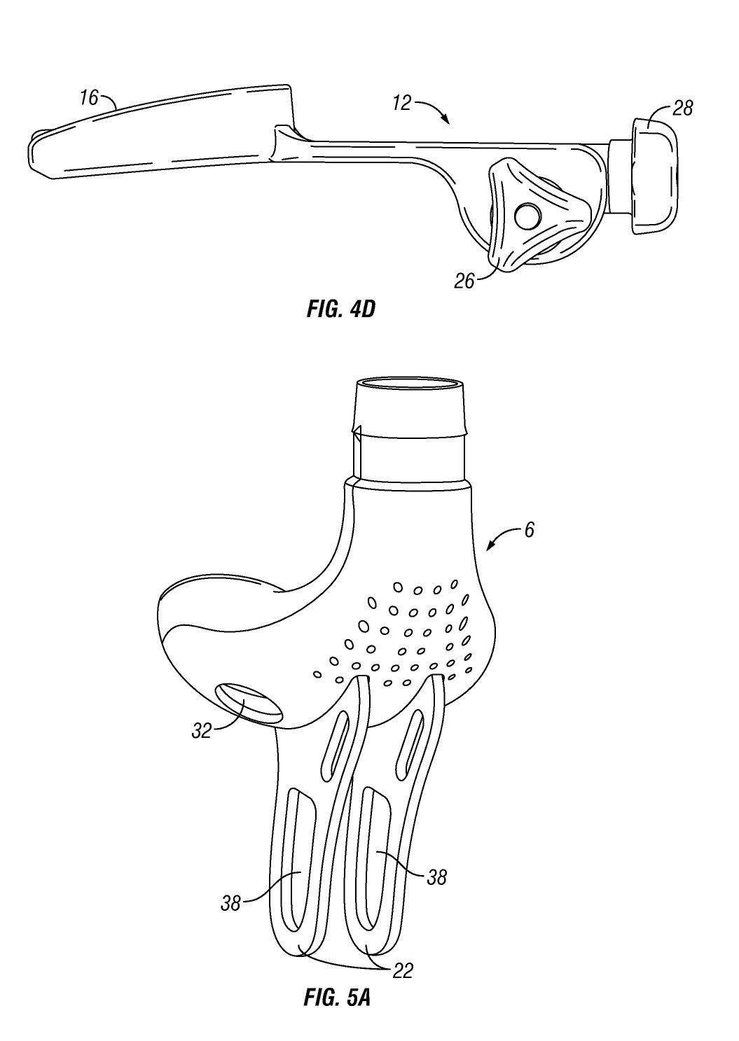

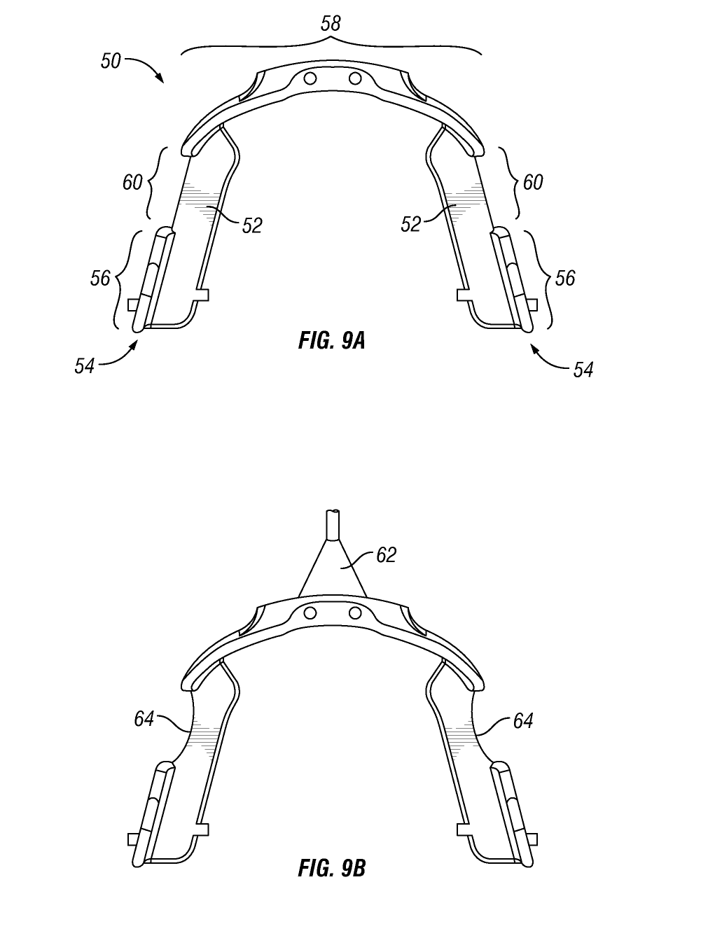

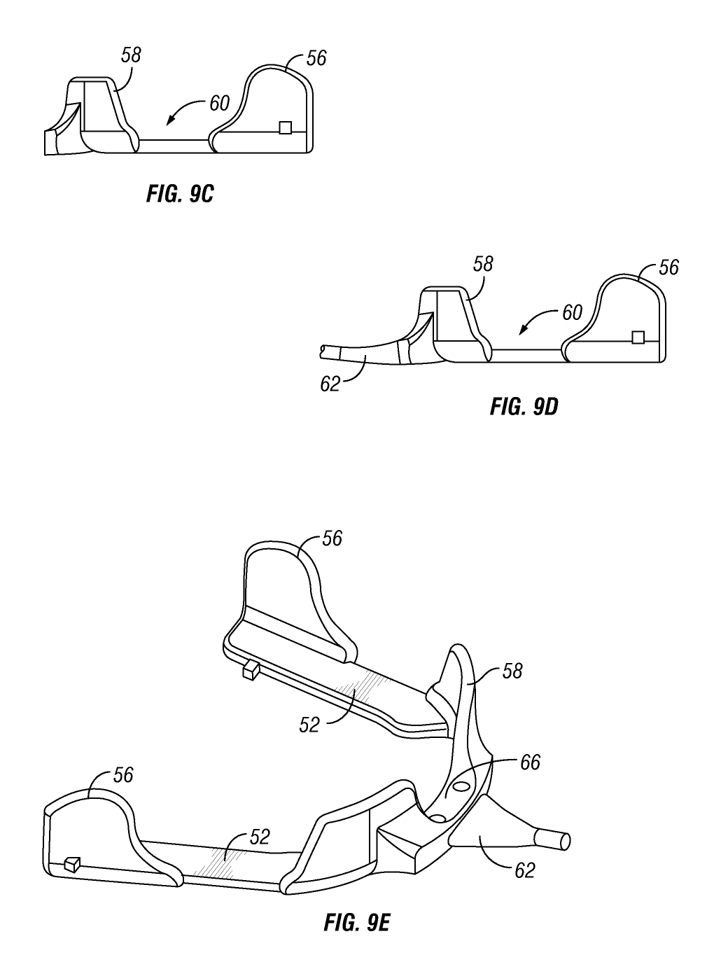

Arches 14 may be any structure configured to engage with the user's dentition. Arches 14 may be oral appliance arches. Arches 14 may include upper arch 16, which is configured to engage with the user's upper dental arch, and lower arch 18, which is configured to engage with the user's lower dental arch. Some embodiments may utilize two arches 14, while other embodiments utilize a single arch 14. Post 12 may be integrally formed with one or more arches 14 (as shown, for example, in FIGS. 4A-4D), or post 40 may be configured to removably couple to one or more arches 14. In embodiments including lower arch 18, which is configured to contact the user's lower dental arch, lower arch 18 may be adjustable relative to upper arch 16 in the anterior-posterior direction. Such adjustment may allow the user's lower jaw to be moved in the anterior direction, which may help to open the user's airway and improve breathing. In some embodiments, arches 14 may include moldable trays that may be custom-fitted to a particular user's dentition. Embodiments using examples of moldable trays are discussed further below with respect to FIGS. 9-11.

Nasal pillows 20 are configured to engage with the user's nostrils. Some embodiments may include nasal pillows 20 having different sizes and lengths. For example, various nasal pillows 20 may have different circumferences, to accommodate different nostril sizes; different lengths, to accommodate different nostril spacing; or any combination thereof. Some embodiments may include a plurality of interchangeable nasal pillows having different shapes and/or sizes. Providing nasal pillows 20 of different sizes and lengths may allow adjustable breathing device 2 to accommodate different nose shapes and/or sizes.

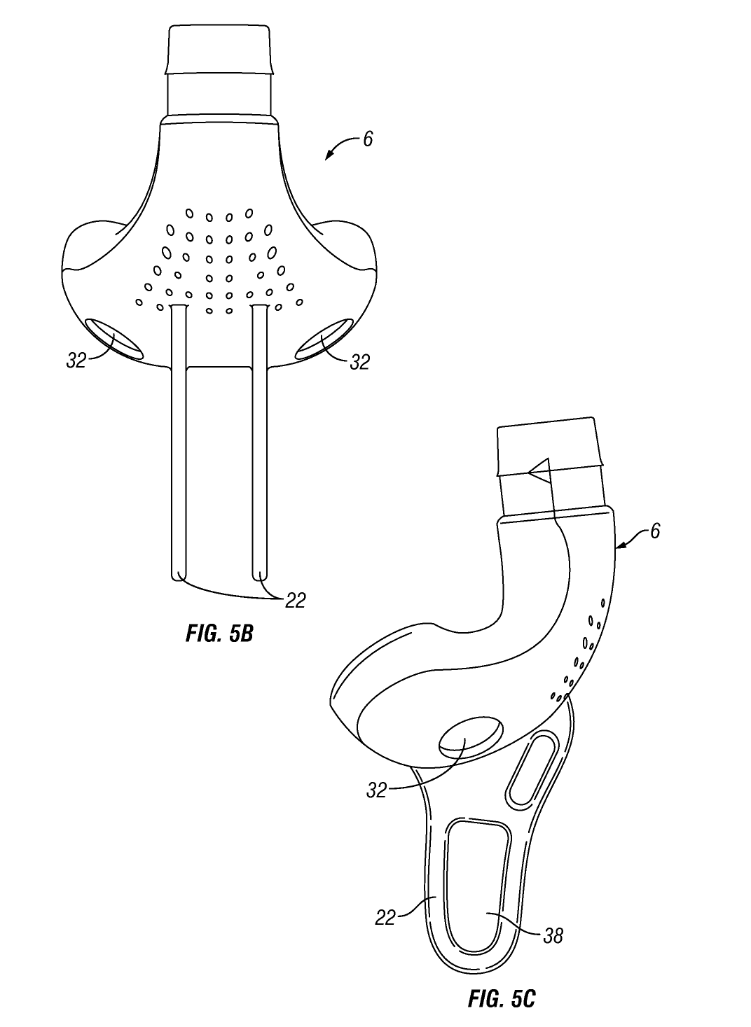

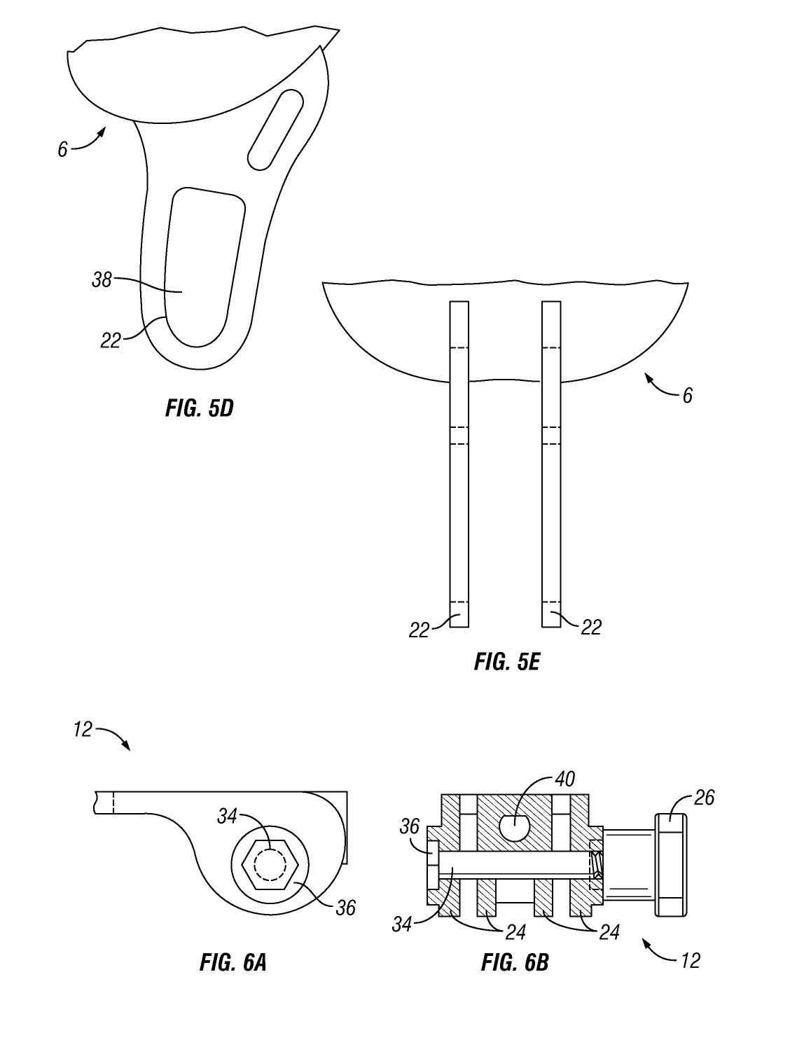

Flanges 22 are operable to engage with post 12 to secure nose mask 6 against post 12. Although illustrated with two flanges 22, other embodiments may use one flange 22, three flanges 22, or any suitable number and configuration of flanges 22. Certain embodiments of nose mask 6 may include one, two, or more than two flanges 22. Flanges 22 may be include polycarbonate, other plastics, or any suitable material. Flanges 22 engage with flanges 24 such that when flanges 22 are engaged with flanges 24, deformation of flanges 22 (and therefore nose mask 6) out of the sagittal plane is substantially prevented. Flanges 22 are discussed further below regarding FIGS. 5A-5E.

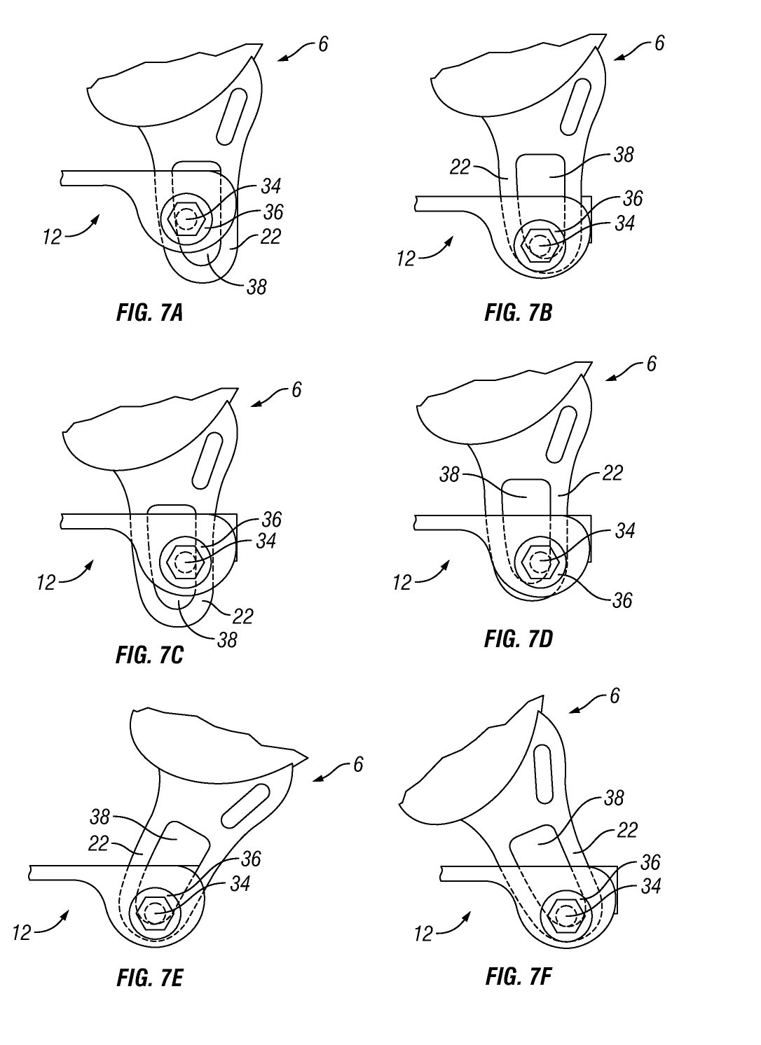

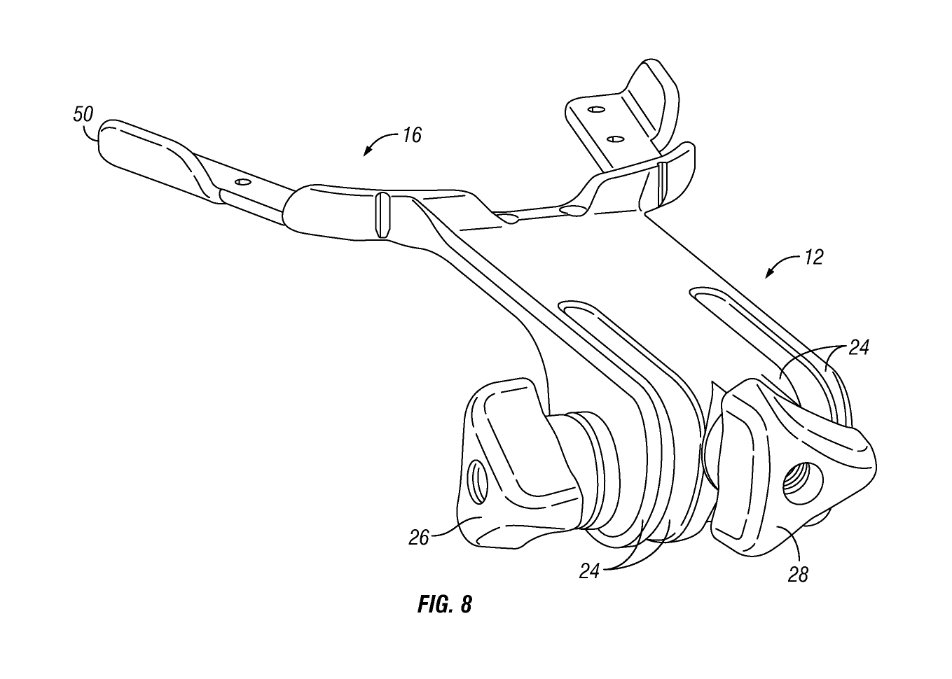

Flanges 24 are operable to be adjustably secured to flanges 22. In certain embodiments, post 12 may include one, two, or more than two flanges 24. Movement of flanges 22 outside of the sagittal plane may be substantially prevented by flanges 24 when flanges 22 are secured to flanges 24. Flanges 24 may have one or more openings through which a fastener 34 (not shown) situated within a slot 39 may pass laterally through flanges 24, as shown, for example, in FIG. 4B. Flanges 22 may be adjustably secured to flanges 24 by the adjustment of tightening knob 26. Flanges 24 and tightening knob 26 may therefore enable adjustment of the superior-inferior position, the anterior-posterior position, and/or the angle within the sagittal plane of flanges 22, and therefore nose mask 6, as illustrated in FIGS. 7A-7F.

Tightening knob 26 may be any suitable mechanism operable to adjustably secure nose mask 6 relative to post 12. While the illustrated embodiment includes a knob, other embodiments may include a clamp, switch, dial, lever, screw, or any suitable adjustment mechanism. Loosening tightening knob 26 may reduce the friction between flanges 24 and flanges 22 such that flanges 22 may be able to move freely within channels 24. Tightening the tightening knob 26 may increase the friction between flanges 24 and flanges 22 such that movement of flanges 22 within channels 24 is substantially restricted. Tightening knob may operate by turning fastener 34 such that the lateral portions of the anterior end of post 12 are brought closer together, thereby narrowing channels 24. Tightening knob 26 may therefore allow nose mask 6 to be adjustably secured relative to post 12, which may allow nose mask 6 to be adjusted to the user's face separately from the adjustment of mouth mask 8.

Tension element adjustor 28 may be any suitable adjustment mechanism operable to adjust the position of one or more arches 14. Tension element adjustor 28 may be a knob, clamp, dial, lever, screw, or any suitable adjustment mechanism. Tension element adjustor 28 may operate to adjust the anterior-posterior position of an arch 14. For example, tension element adjustor 28 may operate to adjust the anterior-posterior position of lower arch 18 relative to upper arch 16. Adjusting lower arch 18 in the anterior direction may bring the user's lower jaw forward, which may help to open the user's airway to improve breathing.

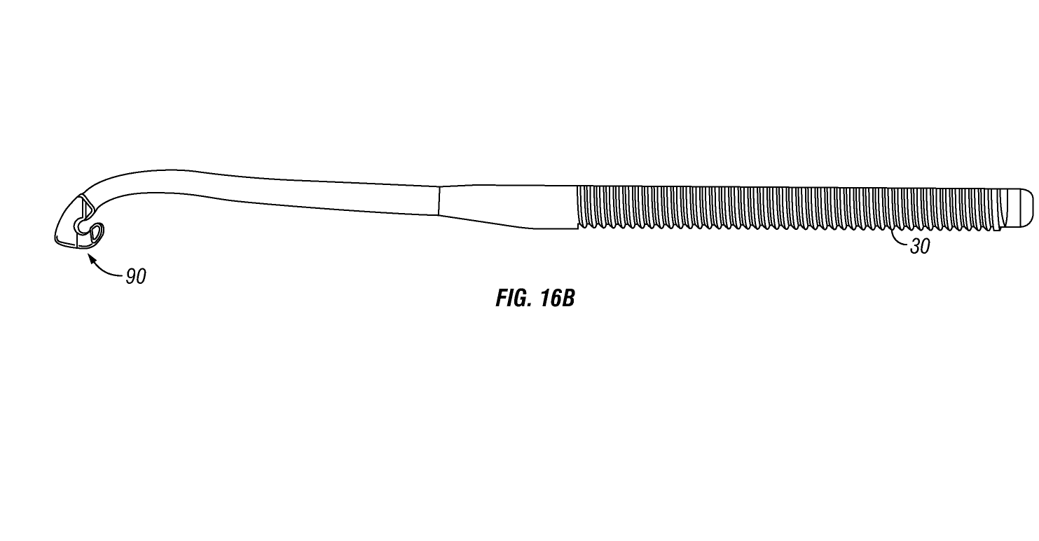

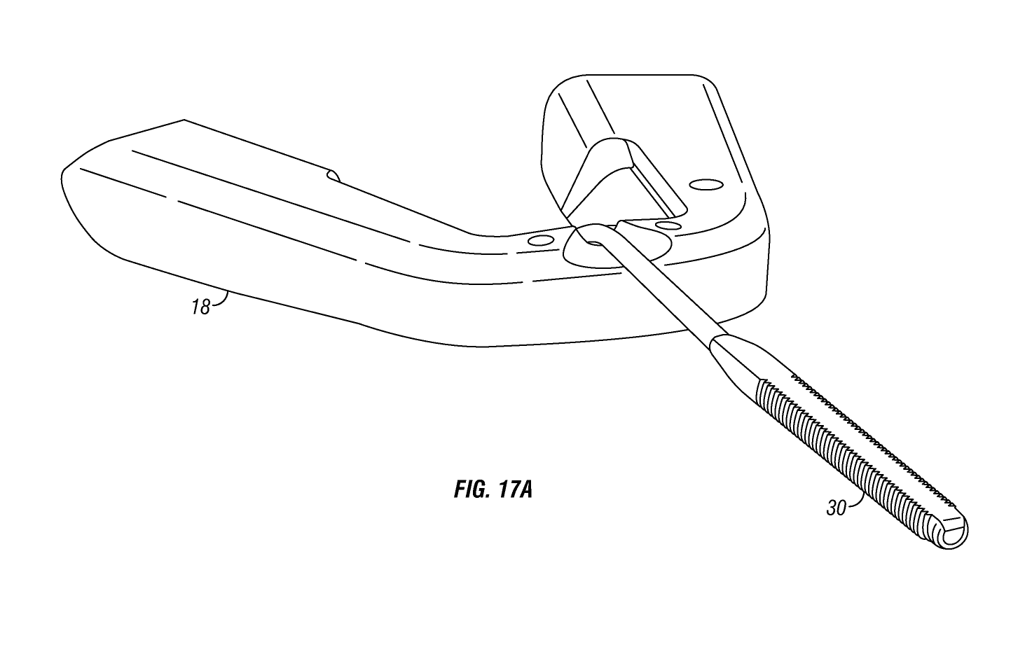

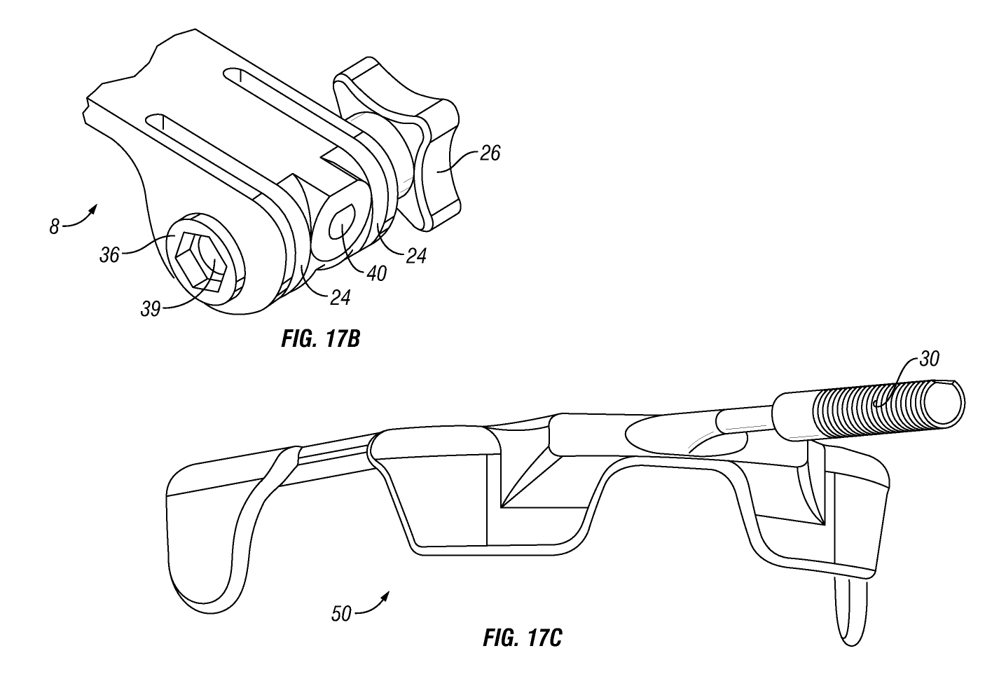

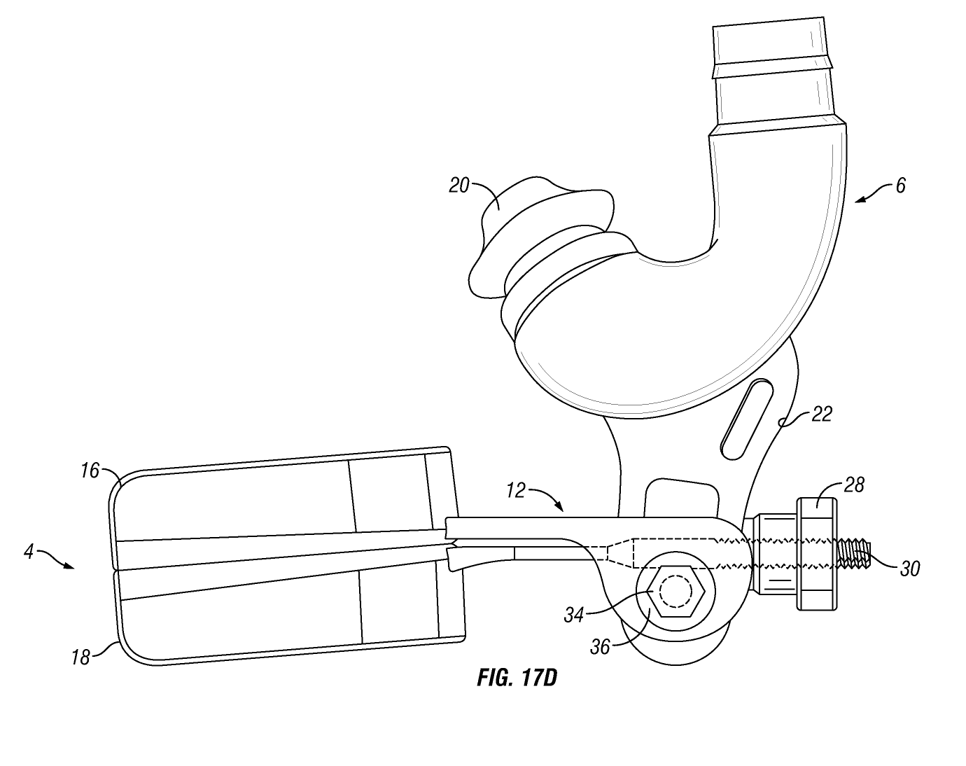

Tension element 30 may be any suitable structure operable to engage with and adjust the position of one or more arches 14. Tension element 30 may engage with one or more arches 14. For example, in some embodiments, tension element 30 is integrally formed with lower arch 18. In other embodiments, tension element 30 may removably connect to lower arch 18. One or more portions of tension element 30 may be threaded to facilitate adjustment of the position of arch 14 when tension element adjustor 28 is actuated. Tension element 30 may pass through a portion of post 12. For example, tension element 30 may pass through tension element slot 40 (not shown), as shown in FIGS. 6B, 17B, and 17D. Tension element 30 is discussed further below regarding FIGS. 16A-16B and 17A-17D. Tension element 30 may be formed from plastic (e.g., nylon), any other suitable material, or any combination thereof. In certain embodiments, tension element 30 is suitably flexible so that, when connected to lower arch 18, the user's lower jaw can be moved slightly, which may improve comfort.

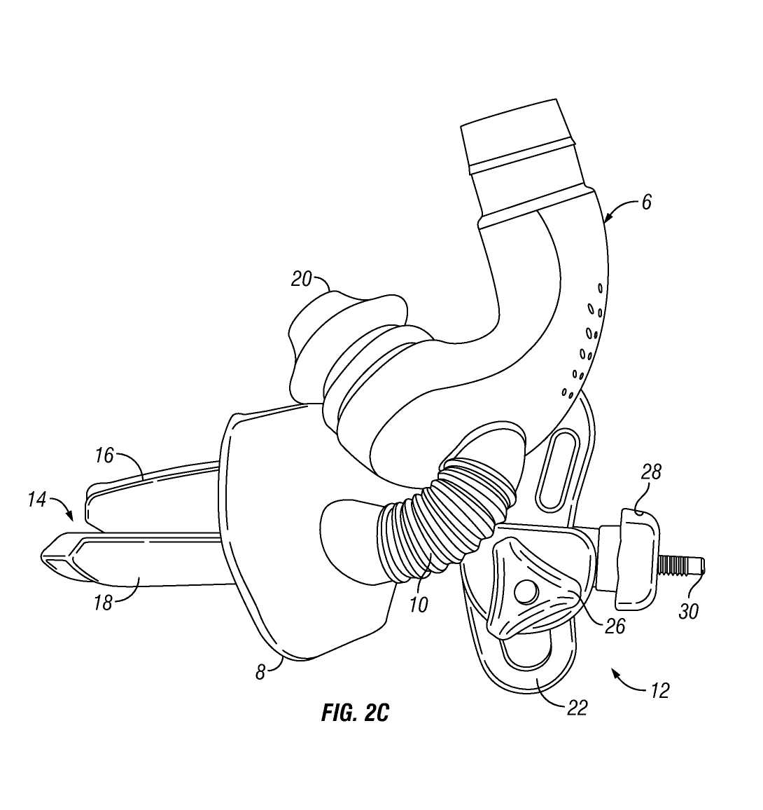

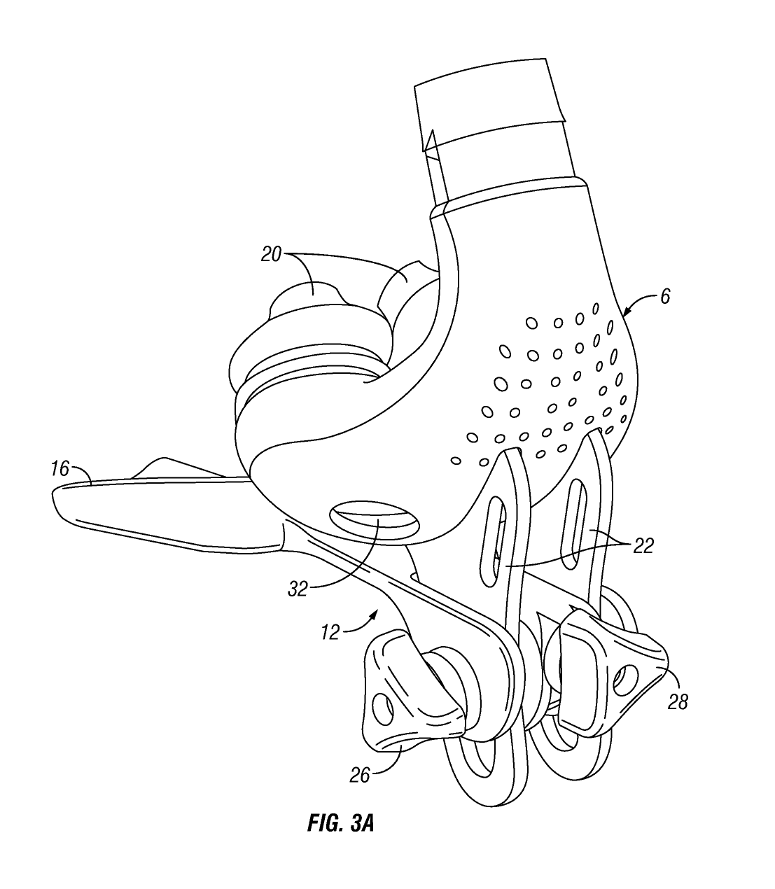

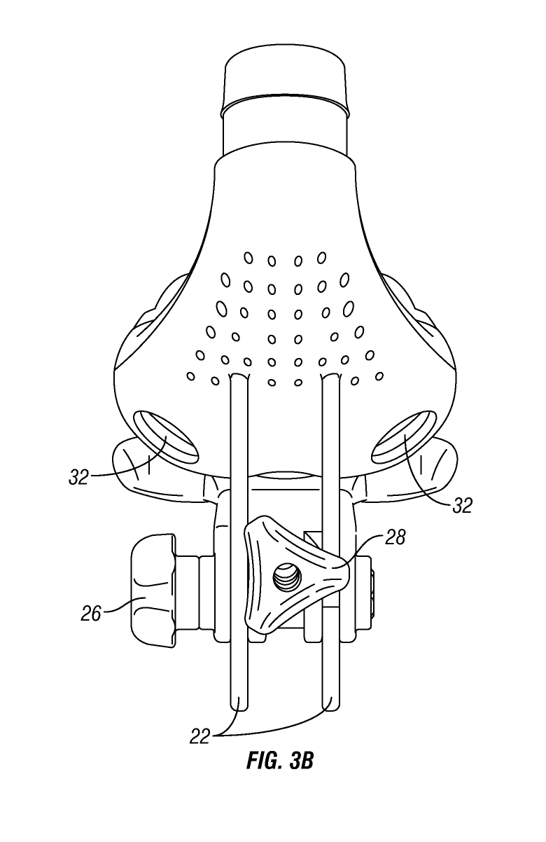

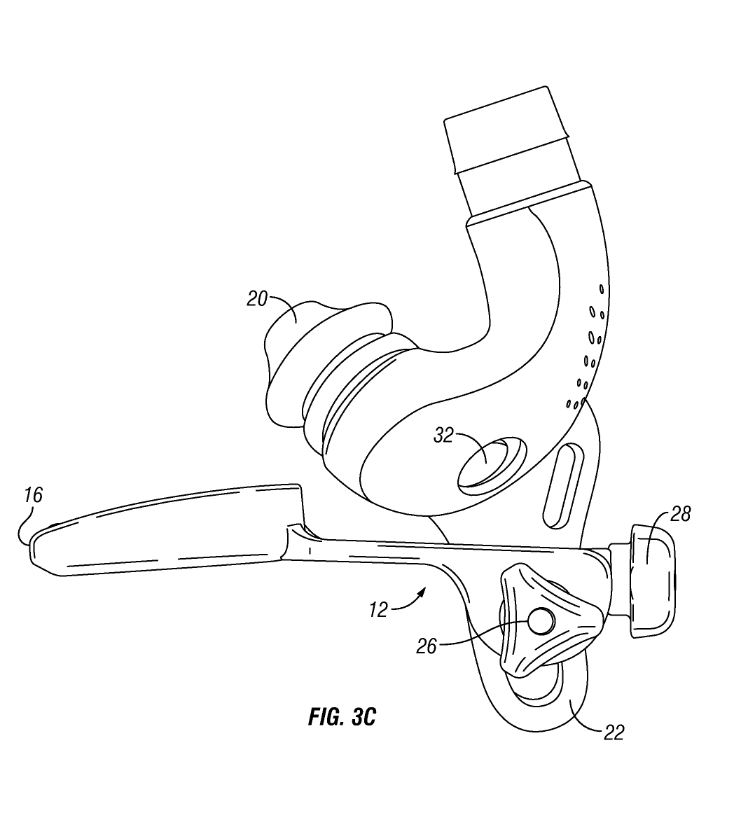

FIG. 3A illustrates examples of a nose mask 6, post 12, and upper arch 16 that may be used in certain embodiments of adjustable breathing device 2. FIGS. 3A, 3B, and 3C depict isometric, front, and side views, respectively. Nose mask 6, which is shown without connector 10, includes openings 32. Post 12 is shown without tension element 30, and lower arch 18 is not depicted.

Openings 32 are configured to engage with connector 10. Openings 32 may be any suitable shape and size to allow a connector to couple to nose mask 6. In embodiments where connector 10 may be removed from mask 6, one or more plugs or caps may be used to fill openings 32 so that air may still be delivered through nose mask 6 even when it is not connected to mouth mask 8 via connector 10. Such embodiments may allow adjustable breathing device 2 to provide different methods of air delivery based on the current needs of the user.

FIGS. 4A-4D illustrate examples of post 12 and upper arch 16 that may be used in certain embodiments of adjustable breathing device 2. FIGS. 4A, 4B, 4C, and 4D depict isometric, top, front, and side views, respectively.

As shown in FIGS. 4A-4D, upper arch 16 may be integrally foci led with post 12. In other embodiments, post 12 may removably couple to upper arch 16 (or lower arch 18) via a coupling mechanism. For example, an arch 14 may include a universal coupler that can attach different types of dental attachments, including post 12. Such universal couplers may include a locking mechanism (such as, for example, a screw, a tab, a groove, a clasp, a clamp, a snap, any suitable fastener, or any combination thereof) that may secure the dental attachment to the arch 14. Embodiments utilizing a post 12 that is integrally formed with an arch 14 may provide simplified fabrication and improved stability. Embodiments utilizing a post 12 that may removably couple to an arch 14 may allow the user to leverage a single arch 14 for multiple uses.

As shown in FIGS. 4B and 4C, post 12 may include fastener 34 and countersink 36. Fastener 34 may include any suitable structure that is operable to restrict the movement of flanges 22 relative to channels 24. In certain embodiments, fastener 34 may be a pin, screw, clasp, or bolt. In the embodiment shown, fastener 34 is a threaded hex-head bolt that engages a hex-head counter sink 36 on one side of post 12 and tightening knob 26 on the other side of post 12. In such embodiments, countersink 36 may prevent rotation of fastener 34 while tightening knob 26 or other appropriate structure is fixing the position of fastener 34. In a particular embodiment, both sides of post 12 may include a countersink 36, such that a user may select to position tightening knob 26 on either side of post 12. This flexibility may make the use of tightening knob 26 equally convenient to left-handed and right-handed users. In alternative embodiments, post 12 may include an additional tightening knob opposite tightening knob 26, enabling adjustment from either side of post 12.

FIGS. 5A-5E illustrate an example nose mask 6 that may be used in certain embodiments of adjustable breathing device 2. FIGS. 5A-5E depict isometric, front, side, side, and front views, respectively.

As shown in FIG. 5A, flanges 22 may include a slot 38. While FIGS. 5A-5E depict two distinct flanges 22, alternative embodiments may have a single flange, multiple flanges, or any other structure which may be secured in place relative to post 12 by fastener 34. In alternative embodiments, slot 38 may have various shapes, sizes, and orientations. In certain embodiments, flanges 22 may be formed and/or molded as one part. In other embodiments, flanges 22 may be formed from multiple parts that may be assembled, clipped, screwed, or overmolded. In various embodiments, flanges 22 may consist of plastic and/or metal such as stainless steel.

FIG. 6A illustrates a side view of a portion of an example post 12 that may be used in certain embodiments of adjustable breathing device 2. As shown in FIG. 6A, the anterior portion of post 12 may include a fastener 34 that engages with countersink 36. Fastener 34 and countersink 36 may include any structure, configuration, and function described above with respect to FIGS. 4B and 4C.

FIG. 6B illustrates a front cross-sectional view of the anterior end of post 12. As shown in FIG. 6B, the anterior end of post 12 may include channel 24, tightening knob 26, fastener 34, countersink 36, and tension element channel 40. The end of fastener 34 may be configured to engage with countersink 34. In certain embodiments, at least a portion of fastener 34 may be threaded. As discussed above, post 12 may include slot 39 (not shown), which may cross laterally through post 12, allowing fastener 34 to pass laterally through post 12 across channels 24. In some embodiments, when flanges 22 are disposed in channels 24, slots 38 of flanges 22 may substantially align with slot 39, allowing fastener 34 to pass through both slot 39 and slots 38. In such an embodiment, tightening knob 26 may be adjusted to increase or decrease lateral force exerted along the long axis of fastener 34. Adjusting tightening knob 26 in such an embodiment may operate to secure flanges 22 in place relative to post 12. Post 12 may thus allow nose mask 6 to be moved into the desired position relative to the user's nose and then fixed in that position by adjusting tightening knob 26.

FIG. 7A-7F illustrate example configurations of an example nose mask 6 and post 12 that may be used in certain embodiments of adjustable breathing device 2. As shown in FIGS. 7A-7F, flange 22 may be capable of moving in the superior-inferior direction relative to post 12, moving in the anterior-posterior direction relative to post 12, and rotating around fastener 34. In alternative embodiments, flange 22 may be capable of moving in the superior-inferior direction relative to post 12 and rotating around fastener 34 but may not be capable of shifting in the anterior-posterior direction relative to post 12. In certain embodiments, flange 22 may be limited to adjustment, rotation, and/or movement within the sagittal plane. In some embodiments, post 12 may include an additional joint that may provide adjustment, rotation, and/or movement in one or more additional directions. In certain embodiments, flange 22 may be adjusted vertically approximately +/−10 mm, though other ranges may be used. For example, other embodiments may have vertical adjustment ranges of between +/−7 mm and +/−14 mm. In certain embodiments, flange 22 may be adjusted in the anterior-posterior direction approximately +/−5 mm, though other ranges may be used. For example, other embodiments may have horizontal adjustment ranges of between +/−3 mm and +/−8 mm. In certain embodiments, when flange 22 has a desired position and orientation, tightening knob 26 may be adjusted to secure flange 22 in place relative to post 12.

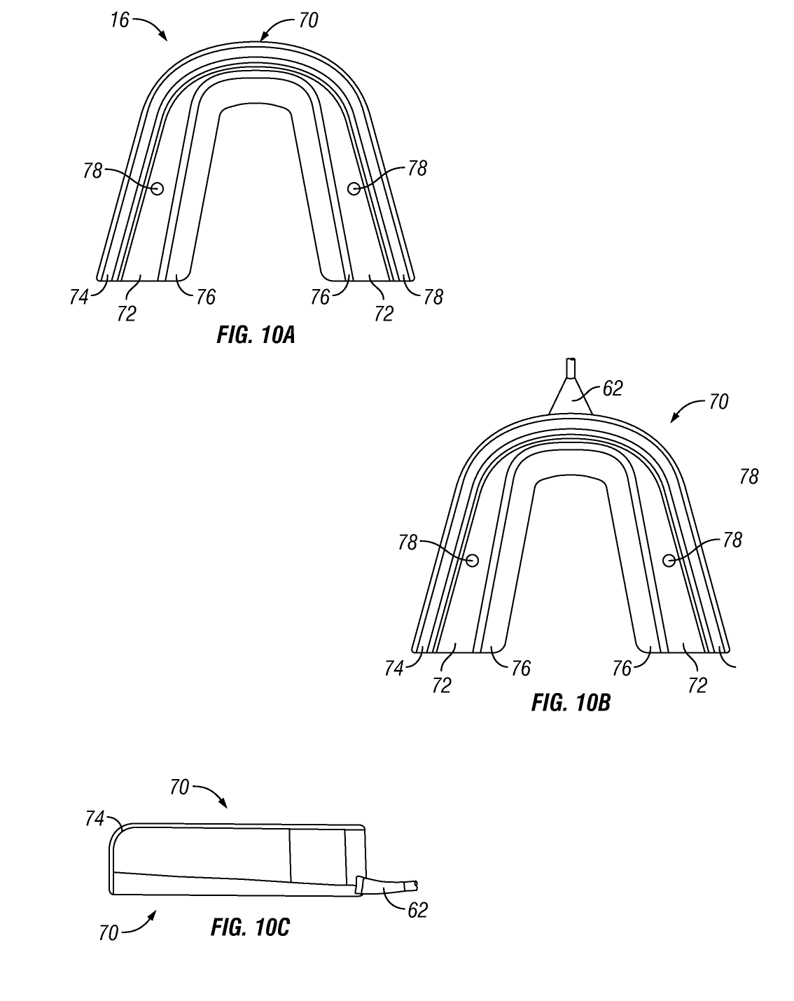

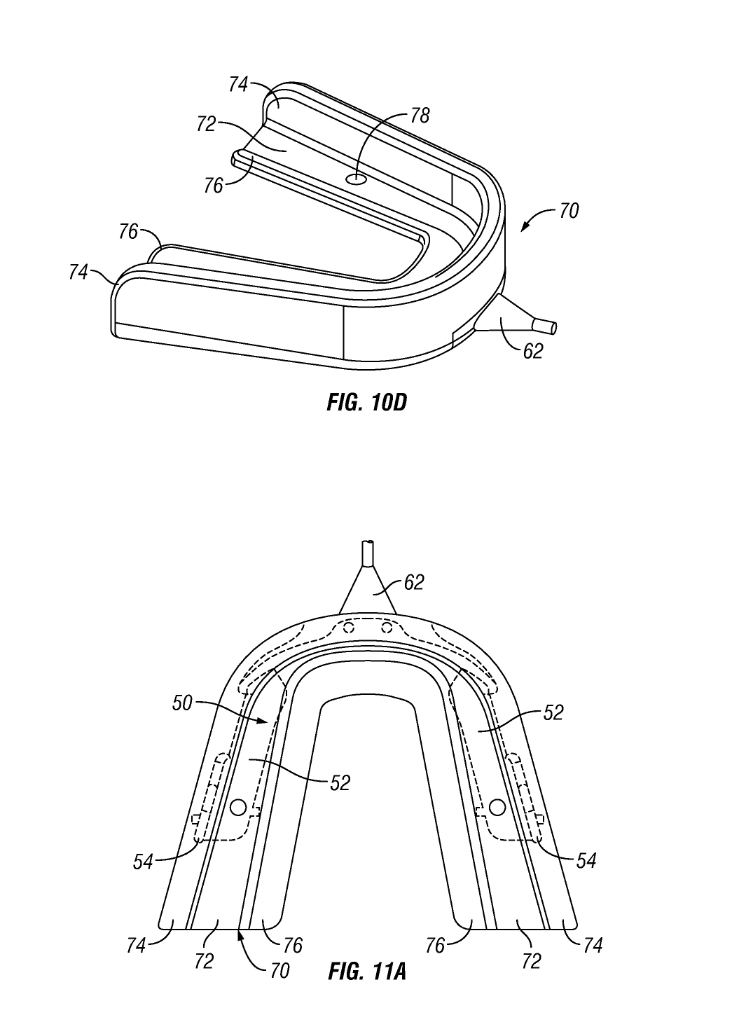

FIG. 8 illustrates an example post 12 and a portion of arch 16 that may be used in certain embodiments of adjustable breathing device 2. In some embodiments, post 12 may be integrally formed with upper arch 16, and upper arch 16 may include an arched frame 50. Arched frame 50 may be at least partially surrounded by a moldable tray 70 (shown in FIGS. 10A-10D). Arched frame 50 is discussed further below with respect to FIGS. 9A-9E.

FIGS. 9A-9E illustrate example arched frames 50 that may be used in certain embodiments of adjustable breathing device 2. Arched frame 50 may include an occlusal surface 52 and a flange 54. In particular embodiments arched frame 50 may be configured to be positioned proximate to a user's dental arch, with occlusal surface 52 positioned proximate to the occlusal surface of the user's dental arch. In certain embodiments, occlusal surface 52 may be contiguous throughout the length of arched frame 50. In alternative embodiments, as shown in FIG. 9A, occlusal surface 52 may not be contiguous throughout the length of arched frame 50. For example, occlusal surface 52 may have a first portion configured to be positioned proximate to the user's left bicuspid and first molar; and have a second portion configured to be positioned proximate to the user's right bicuspid and first molar. As shown in FIG. 9A, in certain embodiments, occlusal surface 52 may not extend to the area proximate to the user's incisors. Certain embodiments in which the occlusal surface 52 is not contiguous throughout the length of arched frame 50 may allow for improved flexibility of arched frame 50. In certain embodiments, arched frame 50 may be capable of flexing inward and/or outward, allowing arched frame 50 to conform to a wider variety of dental arch shapes and sizes. Such embodiments may also improve the ability of arched frame 50 to accommodate the overlap of the user's maxillary and mandibular incisors, allowing the user's jaw to close more fully. In certain embodiments, occlusal surface 52 may have a thickness of approximately 1.5 millimeters, although other thicknesses may be used.

Flange 54 may run along the labial edge of arched frame 50. In certain embodiments, flange 54 may be contiguous throughout the length of arched frame 50. In alternative embodiments, as shown in FIG. 9A, flange 54 may not be contiguous throughout the length of the arched frame. For example, flange 54 may include a distal flange portion 56 and a mesial flange portion 58, separated by a flange recess 60. In certain embodiments, because flange 54 is positioned proximate to the labial surface of the user's dental arch, it may be pushed outward by the labial surface of the user's dental arch when arched frame 50 is inserted into the user's mouth during the molding process, allowing arched frame 50 to automatically flex and align with the user's dental arch, which may improve the ability of arched frame 50 to accommodate different dental arch sizes and shapes. In certain embodiments, flange recesses 60 may allow for improved flexibility of arched frame 50. In addition, when arched frame 50 is used with deformable material, flange recesses 60 may allow the deformable material to form an improved mold of the user's teeth. In some embodiments, flange recess 60 may improve the user's ability to press moldable material against their teeth during the molding process, which allow for improved dental molds. In certain embodiments, mesial flange portion 58 may allow for an improved mold when arched frame 50 is pressed toward the user's teeth during fitting. As shown in FIG. 9A, in certain embodiments mesial flange portion 58 may have a thickness greater than that of distal flange portion 56. For example, mesial flange portion 58 may have a thickness of approximately 3 millimeters and distal flange portion 56 may have a thickness of approximately 1.5 millimeters, although other thicknesses may be used. In such embodiments, the greater thickness of mesial flange portion 58 may improve the stability of arched frame 50 during flexion and may provide a more secure anchor point for other attached structures, such as anterior structure 62, shown in FIG. 9B.

FIG. 9B illustrates another example arched frame 50 having occlusal surface 52, flange 54, and an anterior structure 62. As shown in FIG. 9B, in certain embodiments, occlusal surface 52 may include an occlusal surface recess 64. When arched frame 50 is used with deformable material, occlusal surface recess 64 may allow the deformable material to form a closer mold of the user's teeth. As shown in FIG. 9B, certain embodiments may include anterior structure 62 which extends forward from mesial flange portion 58 in an anterior direction. In certain embodiments, anterior structure 62 may be fixed to arched frame 50, while in other embodiments anterior structure 62 may removably coupled to arched frame 50. FIGS. 9C and 9D illustrate side views of example arched frames 50 having occlusal surface 52 and flange 54 with distal flange portion 56, flange recess 60, and anterior structure 62.

FIG. 9E illustrates an isometric view of an example arched frame 50. As shown in FIG. 9E, arched frame 50 may include anterior structure 62, occlusal surface 52 that is not contiguous throughout the length of arched frame 50, and flange 54 that includes a distal flange portion 56, a flange recess 60, a mesial flange portion 58, and a mesial flange recess 66. As shown in FIG. 9E, flange 54 may include mesial flange recess 66 located approximately at the midline of arched frame 50. In such embodiments, mesial flange recess 66 may allow for improved conformity with the shape of the user's mouth. In certain embodiments, the distal ends of arched frame 50 may extend approximately to the user's first molar when the frame is inserted into the user's mouth. In alternative embodiments, arched frame 50 may extend to the user's second molar or to the user's third molar.

In some embodiments, arched frame 50 may be primarily composed of a substantially rigid material, such as polycarbonate or any other material providing substantial rigidity while allowing moderate flexion. Embodiments utilizing polycarbonate may improve the ability of arched frame 50 to adhere to moldable tray 70. In certain embodiments, arched frame 50 may be composed of a material whose form does not substantially changed when heated to the temperature required to soften the moldable material of moldable tray 70. For example, in some embodiments, arched frame 50 may be composed of a material that substantially maintains its shape when heated up to at least 100 degrees Celsius. Such materials may include polycarbonate, Nylon, acrylonitrile butadiene styrene (ABS), or polyethylene. In certain embodiments, arched frame 50 may be composed of a semi-flexible material, for example liquid silicone rubber (LSR), approximately having a Shore 30-90 hardness, although this particular hardness is not required.

FIGS. 10A through 10D illustrate example moldable trays 70. As shown in FIG. 10A, moldable tray 70 may include an occlusal surface 72, an outer rim 74, an inner rim 76, and recesses 78. Occlusal surface 72 may be configured to be placed proximate to the occlusal surface of a user's dental arch. Outer rim 74 may be configured to be positioned proximate to the labial surface of a user's dental arch. In certain embodiments, inner rim 76 may be configured to be positioned proximate to the lingual surface of a user's dental arch. In certain embodiments, inner rim 76 may lie mostly flat relative to the plane of occlusal surface 72 or may angle upward slightly. Such embodiments may make moldable tray 70 easier to slide into the user's mouth. In certain embodiments, inner rim 76 may be capable of being pushed upward or downward to engage with the lingual surface of the user's dental arch during the molding process.

As shown in FIG. 10A, in certain embodiments, outer rim 74 may have a thickness greater than that of inner rim 76. For example, in certain embodiments, outer rim 74 may have a thickness of approximately 3 millimeters, while inner rim 76 may have a thickness of approximately 2 millimeters, although these dimensions are not required. Reduced thickness of inner rim 76 may allow moldable tray 70 to take up less space in the inner mouth area behind the teeth, which may allow the user to breath, swallow, and speak more easily and experience greater comfort. Reduced thickness of inner rim 76 may also help obviate any need to offer multiple sizes of moldable tray 70 and arched frame 50. In certain embodiments, reduced thickness of inner rim 76 may allow other medical and/or dental devices to be more easily inserted into the user's mouth. In certain embodiments, as shown in FIG. 10D, inner rim 76 may be shorter than outer rim 74. A shorter inner rim 76 may allow for easier insertion of moldable tray 70 into the user's mouth. A shorter inner rim 76 may also reduce the amount of moldable material in the inner mouth area, which may provide additional advantages as described above. In certain embodiments, distal portions of moldable tray 70 may have a reduced height, which may improve the fit of moldable tray 70 in the user's mouth.

As shown in FIG. 10A, in certain embodiments, occlusal surface 72 may have one or more recesses 78, which may result from clamping or otherwise holding in place arched frame 50 during an overmolding process. In certain embodiments, arched frame 50 illustrated in FIGS. 8A-8E may have a corresponding recess, which may allow for improved clamping and alignment during the manufacturing process.

FIG. 10B shows another example moldable tray 70 having occlusal surface 72, outer rim 74, and inner rim 76. As shown in FIG. 10B, in certain embodiments, moldable tray 70 may further include an anterior structure 62. It should be appreciated that in certain embodiments the optional anterior structure 62 may be fixed to moldable tray 70 or it may be removably coupled to moldable tray 70.

In certain embodiments, moldable tray 70 may be composed of a material that can be heated to a temperature at which the material softens and becomes capable of being molded to a different shape. In certain embodiments, the material can be heated in hot water. In some embodiments, the temperature range at which the material softens may be approximately 40-80 degrees Celsius, although materials with other softening ranges may be used. In a particular embodiment, the target softening temperature may be approximately 60 degrees Celsius. In certain embodiments, this material may be a thermoplastic. Such thermoplastic materials may be heated to a temperature at which the thermoplastic becomes soft and moldable, at which point it may be molded to the shape of at least a portion of a user's dental arch and become at least temporarily fixed in that shape.

As one example, moldable tray 70 may comprise a polycaprolactone polymer or other aliphatic polyester. In particular embodiments, the thermoplastic material may comprise a cross-linked polycaprolactone reinforced with an aramid fiber such as the short length aramid fiber sold by Dupont under the brand name Kevlar®. In certain embodiments, using polycaprolactone combined with Kevlar® may allow moldable tray 70 to soften at low temperatures and set hard at temperatures of approximately 60 degrees Celsius. In certain embodiments, using polycaprolactone combined with Kevlar® may improve the hardness of moldable tray 70 following the molding process, which may improve the ability of moldable tray 70 to hold its shape when being used to adjust the user's jaw position and/or hold a mask or other breathing device in place. In certain embodiments, this increased hardness may also improve the ability of moldable tray 70 to hold its shape for longer periods of time. For example, in certain embodiments, this may allow moldable tray 70 to substantially hold its shape for periods longer than approximately 1 month, though such period is not required. Using polycaprolactone combined with Kevlar® may also allow for thinner embodiments of moldable tray 70, which may allow moldable tray 70 to take up less space in the user's mouth. Examples of polycaprolactone combined with an aramid fiber, including Kevlar® and a variety of other fibers, are described in U.S. application Ser. No. 11/368,991, publication number U.S. 2007/0004993 A1, which is incorporated herein by reference. Such embodiments may provide an improved moldable material that better maintains its form when heated, providing increased viscosity which may prevent the material from flowing excessively around the user's teeth and/or getting stuck on the user's teeth during the molding process. Such embodiments may also possess increased strength after molding.

In certain embodiments, the thermoplastic material may be cross-linked by radiation, which may create cross-linking of certain molecules to improve the material's shape retention characteristics and/or make the material better able to return to its original shape after reheating. In certain embodiments, radiation may be applied after moldable tray 70 has been overmolded with arched frame 50, but before being molded to the user, though this is not required. Cross-linking by radiation may provide improved shape-retention characteristics, such that moldable tray 70 may be better able to return to its pre-custom-molding shape under certain conditions. Cross-linking by radiation is further described in U.S. Pat. No. 5,415,623, which is incorporated herein by reference.

Cross-linking certain molecules of moldable tray 70 by applying radiation may allow moldable tray 70 to be shaped to conform to the user's dentition while having improved memory, such that moldable tray 70 may be better able to return to its pre-custom-molded shape under certain conditions. Moldable tray 70 may have a first shape that may be partially or substantially defined by the memory. The term “memory” is used herein to describe the characteristic of plastics or polymeric materials to return to an original shape after distortion from the original shape into a second shape. Distortion from the original shape to the second shape may occur, for example, during the custom-molding processes described above with respect to moldable tray 70. Returning to the original shape may occur under certain conditions, such as, for example, heating to a temperature lower than the melting temperature of the plastic yet high enough to cause deformation of the plastic towards its preform shape. The temperature necessary to activate the memory may be the same or similar to temperature used to mold the material into its second shape. The first (or “preform”) shape can be imparted memory by various means depending upon the particular plastic or polymeric material used. For example, memory can be imparted to the first shape by mechanical forming, chemical radiation or other cross-linking, predetermined stretching, molding in place, or other known procedures.

Certain plastics materials that are capable of having a memory imparted to them include, but are not limited to, caprolactone polymer (sometimes known as oxepanone polymer), which may be a polymer having the formula:

H[0(CH2)5CO]xO(CH2)4-H[O(CH2)5CO]yO

Caprolactone polymer may also be initiated with butane diol. Other materials may include poly(ethylene adipate), poly(epsilon-caprolactone), polyvinyl stearate cellulose acetate butyrate, ethyl cellulose, any suitable material, or any suitable combination thereof. In certain embodiments, moldable tray 70 may be formed from a polymeric material, such a caprolactone polymer, which has a molecular weight of about 50,000, a melting point of about 58 to 60 degrees Celsius, a specific gravity of 1.10 at 20 degrees Celsius, and a viscosity of 1.5 million centipoises at 100 degrees Celsius. In some embodiments, moldable tray 70 may be formed from an extruded sheet of low temperature plastic stock material using, for example, the materials listed above or any suitable combination thereof.

Memory may be imparted to the premold shape of moldable tray 70 by cross-linked certain molecules with radiation, which may cause the material to partially or substantially return to its preform shape under certain conditions (such as, for example, heating moldable tray 70 to temperatures of 140 to 200 degrees Fahrenheit in embodiments where moldable tray 70 is formed from caprolactone). In some embodiments, moldable tray 70 may be irradiated at doses of between 5 to 40 megarads, which may be performed, for example, using a cobalt gamma irradiator. In some embodiments, values of approximately 10 megarads may impart sufficient memory to the material of moldable tray 70. In some embodiments, using more than 40 megarads may cause embrittlement of moldable tray 70. In some embodiments, 10-15 megarads may be preferred. After cross-linking, moldable tray 70 may be formed into its second shape by heating moldable tray 70 and molding it to conform to the user's dentition as described above. Following molding, moldable tray 70 may remain stiff and substantially unyielding at ambient temperatures. However, under certain conditions, such as when the temperature exceeds 140 degrees Fahrenheit, moldable tray 70 may tend to return to its preform shape.

In certain embodiments, the material may exhibit slight shrinkage after being molded to the user's dental arch. In particular embodiments, such shrinkage may be less than 1%. Slight shrinkage of the material following the molding process may allow for improved fit with the user's dental arch. In some embodiments, slight shrinkage of the material following the molding process may allow moldable tray 70 to have a “snap” fit with the user's dental arch.

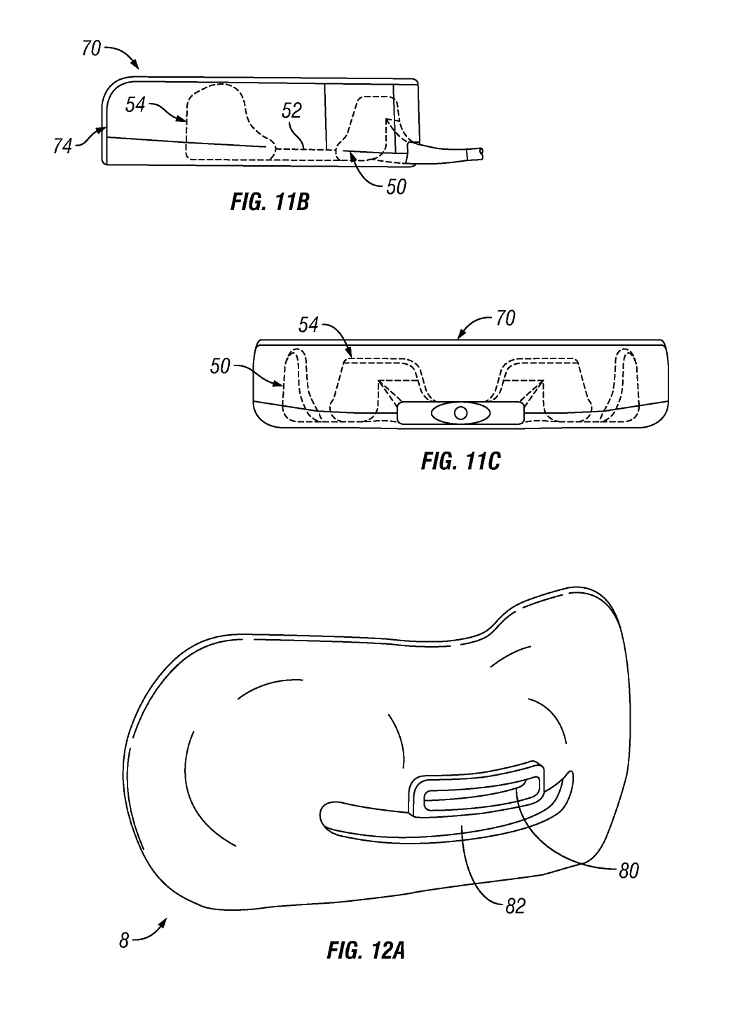

FIGS. 11A-11C illustrate an example arched frame 50 and moldable tray 70 that may be used in certain embodiments of an arch 14. As shown in FIGS. 11A-11C, arch 14 may include a moldable tray 70 substantially surrounding an arched frame 50. As shown in FIG. 11A, example moldable tray 70 may include occlusal surface 72, outer rim 74, and inner rim 76; and arched frame 50 may include occlusal surface 52 and flange 54. As shown in FIG. 11A, in certain embodiments, the labial edge of outer rim 74 may extend outward beyond the labial edge of flange 54. The lingual edge of inner rim 76 may also extend inward beyond the labial edge of occlusal surface 52. As seen in FIG. 11B, in certain embodiments, the distal end of moldable tray 70 may extend distally beyond the distal end of arched frame 50. In certain embodiments, the distal end of arched frame 50 may extend approximately to the user's first molar, while the distal end of moldable tray 70 extends approximately to the user's second or third molar. In alternative embodiments, the distal end of arched frame 50 may extend approximately to the user's second molar, while the distal end of moldable tray 70 extends approximately to the user's third molar. In still other embodiments, the distal ends of arched frame 50 and moldable tray 70 may be approximately coextensive.

In certain embodiments, when moldable tray 70 is oriented for placement on, for example, a user's maxillary arch, the superior surface of outer rim 74 may extend beyond the superior surface of flange 54 by approximately 2.5 millimeters while the inferior surface of moldable tray 70 may extend below the inferior surface of arched frame 50 by approximately 1.5 millimeters, although these dimensions are not required. In certain embodiments, moldable tray 70 may extend outward beyond the labial edge of arched frame 50 by approximately 1.5 millimeters, though other dimensions are possible. In certain embodiments, moldable tray 70 may extend inward beyond the lingual edge of arched frame 50 by approximately 1.5 millimeters, though other dimensions are possible.

In certain embodiments, flange 54 may help maintain the shape of outer rim 74. Moldable trays that substantially surround an arched frame may allow for reduced bulk between a user's incisors when the tray(s) are inserted into the user's mouth. By providing moldable trays with less material between the user's incisors, certain embodiments may allow users to close their mouths further with the trays inserted, which may improve comfort and/or effectiveness. Furthermore, moldable trays that substantially surround arched frame may allow for mouth pieces where only the moldable material touches the inner surfaces of the user's mouth, such as the user's gums, lips, and tongue. Such moldable trays may also allow for improved molding to the user's front teeth. Having arched frame 50 substantially surrounded by moldable tray 70 may also reduce the chances of damage to arched frame 50 and may help hold any broken pieces of arched frame 50 in place, preventing any such broken pieces from contacting the user's mouth or entering the user's airway.

In certain embodiments, arched frame 50 may include apertures in occlusal surface 52 and/or flange 54, though such apertures are not required. Such apertures may allow the moldable material to flow through arched frame 50 during the molding process, which may provide greater stiffness following the molding process and may allow for improved alignment of arched frame 50 with moldable tray 70.

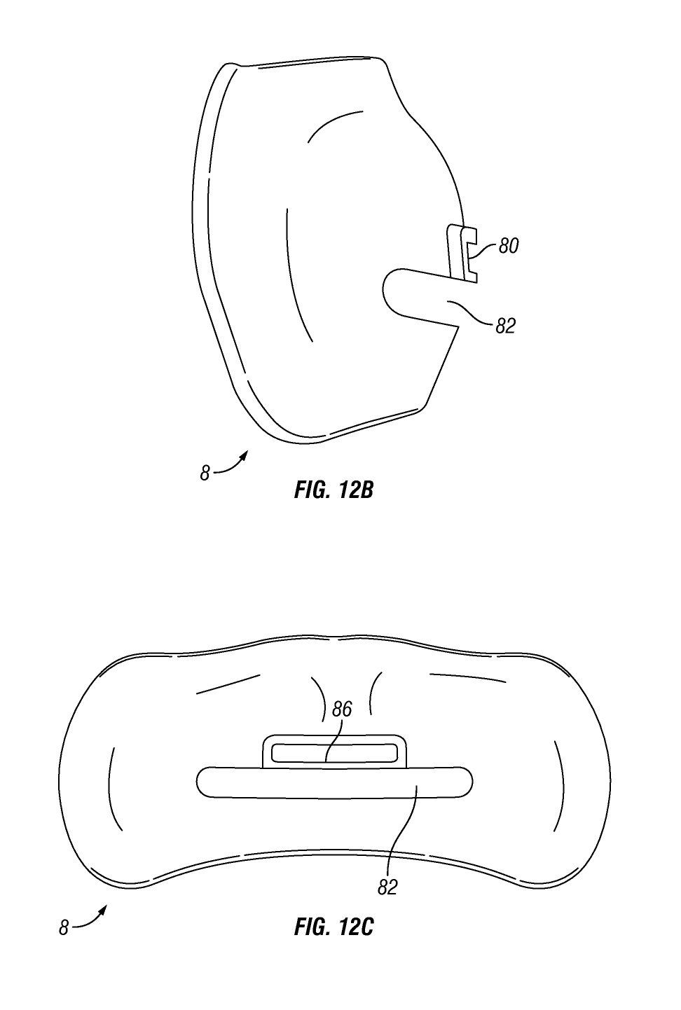

FIG. 12A-12C illustrate an example mouth mask 8 that may be used in certain embodiments of adjustable breathing device 2. FIG. 12A-12C depict isometric, side, and front views, respectively. Mouth mask 8 may include sealing portion 80 and connector opening 82.

Sealing portion 80 may be any suitable structure configured to receive and form a substantial seal around post 12. In some embodiments, sealing portion 80 may be thicker than the surrounding material of mouth mask 8 to provide improved resiliency. In certain embodiments, post 12 may be received by mouth mask 8 by stretching the opening of sealing portion 80 around post 12. In other embodiments, mouth mask 8 may include a slit (not shown) passing from the outer edge of mouth mask 8 to the inner edge of the sealing portion. This slit may be pulled open to allow post 12 to pass through mouth mask 8 and into the opening defined by sealing portion 80, and the slit may then seal substantially shut so that mouth mask 8 may still provide a substantially air-tight seal around the user's mouth.

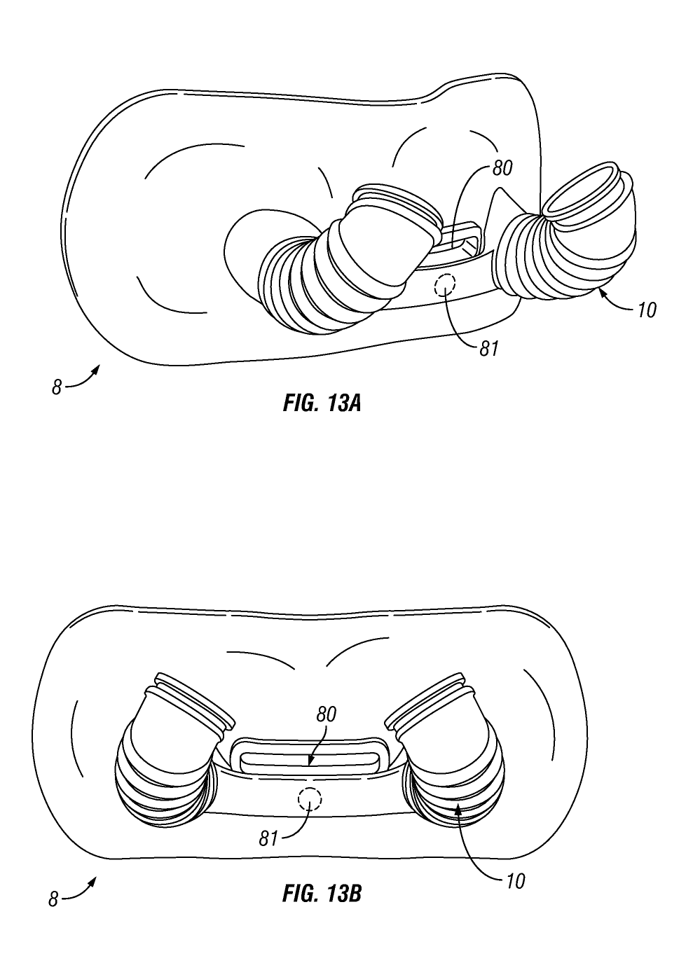

Connector opening 82 may be any suitable opening configured to engage with connector 10. When connector 10 is coupled with mouth mask 8, gas flowing through the interior space of connector 10 may pass through connector opening 82 and into the interior space of mouth mask 8. In some embodiments, when connector 10 is coupled with connector opening 82, a substantially air-tight may be formed between mouth mask 8 and connector 10. Mouth mask 8 may have any suitable number, shape, or size of connector openings 82. When mouth mask 8 is not coupled with connector 10, one or more plugs or caps may cover connector opening 82 to allow mouth mask 8 to provide a substantially air-tight seal around the user's oral cavity. Mouth mask 8 is shown coupled with connector 10 in FIGS. 13A and 13B.

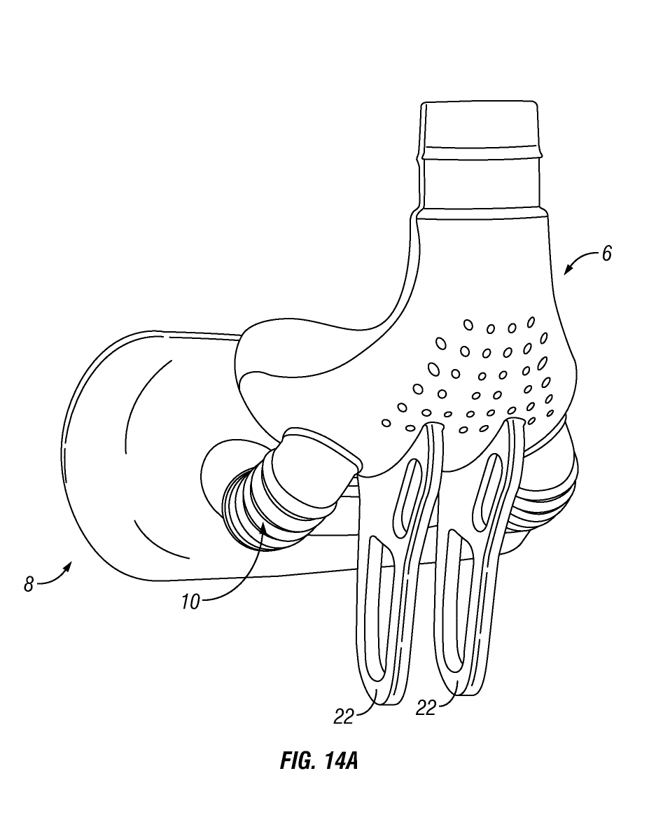

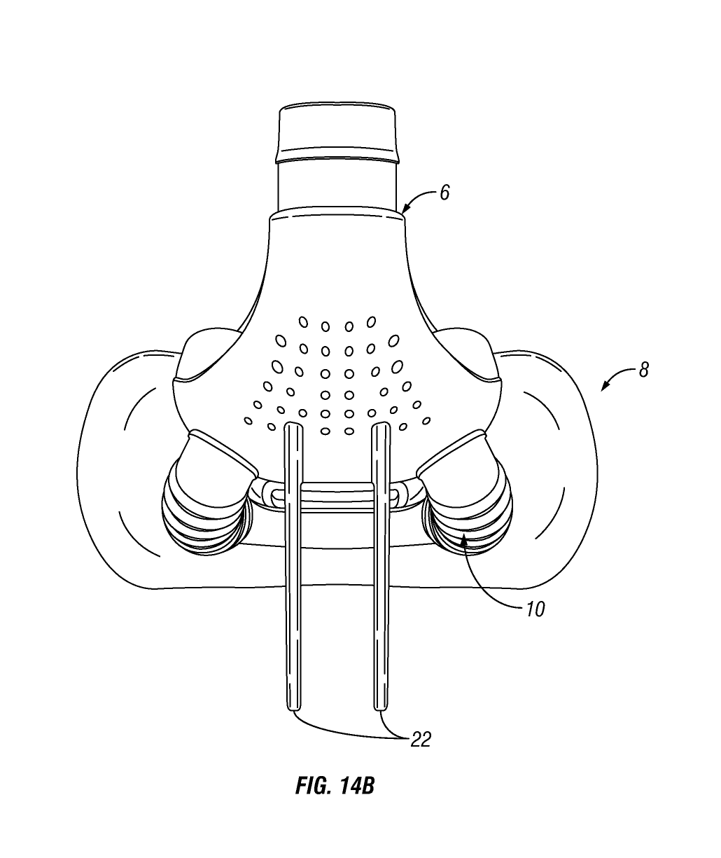

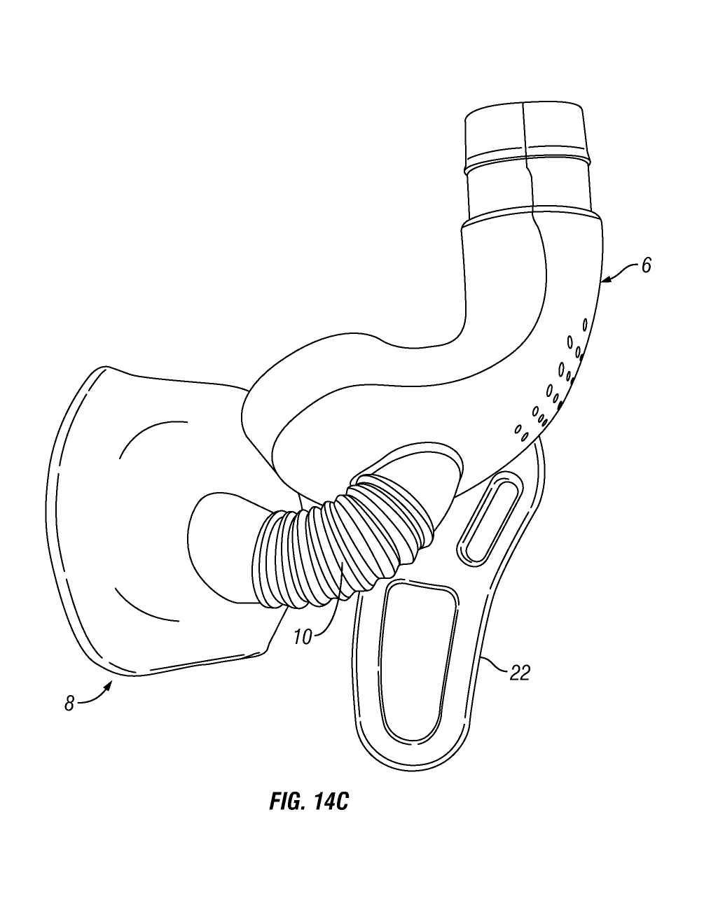

FIGS. 13A and 13B illustrate an example mouth mask 8 and connector 10 that may be used in certain embodiments of an adjustable breathing device. FIGS. 13A and 13B depict isometric and front views, respectively. As shown in FIGS. 13A and 13B, connector 10 may engage with mouth mask 8 at connector opening 82 (shown in FIGS. 12A and 12B). The other end of connector 10 may also couple to nose mask 6 such the interior spaces of nose mask 6 and mouth mask 8 are connected by the interior space of connector 10, as shown in FIGS. 14A-14C. Connector 10 may also include opening 81. Opening 81 is configured to receive tension element 30. In some embodiments, opening 81 may be self-sealing such that opening 81 is closed when tension element 30 is not engaged. In other embodiments, opening 81 may be closed with a seal, plug, flap, or any suitable component to maintain a seal over the user's mouth when tension element 30 is not present. Such embodiments may provide dynamic configurability when optional elements, such as tension element 30, are used to adjust lower arch 18 or perform other functions.

FIGS. 14A-14C illustrate examples of nose mask 6, mouth mask 8, and connector 10 that may be used in certain embodiments of adjustable breathing device 2. FIGS. 14A-14C depict isometric, front, and side views, respectively. When connector 10 is connected to both nose mask 6 and mouth mask 8, as shown in FIGS. 14A and 14B, the interior spaces of nose mask 6 and mouth mask 8 are connected by the interior space of connector 10. This configuration may allow gas delivered to one mask to be conveyed to the other mask, which may allow gas to be delivered via both nose mask 6 and mouth mask 8 without requiring separate connections to the gas delivery device. Furthermore, because each mask is separately adjustable relative to relevant portion of the user's face, the same set of masks may be configured for use by users with a wide variety of facial features.

FIG. 15 illustrates an example mouth mask 8 and post 12 that may be used in certain embodiments of adjustable breathing device 2. As shown in FIG. 15, sealing portion 80 of mouth mask 8 may receive and form a substantial seal around a portion of post 12. Frictional force between sealing portion 80 and post 12 may also operate to secure mouth mask 8 in place relative to post 12. Other embodiments may use one or more clamps, screws, or any suitable fastener to secure mouth mask 8 relative to post 12. Mouth mask 8 may therefore be adjusted by sliding post 12 through sealing portion 80 until mouth mask 8 is in the desired position against the user's face. In some embodiments, post 12 may include one or more ridges or notches (not shown) that may be used to provide distinct adjustment points for mouth mask 8.

FIGS. 16A and 16B illustrate an example tension element 30 that may be used in certain embodiments of adjustable breathing device 2. In some embodiments, tension element 30 may be integrally formed with an arch 14. In other embodiments, tension element 30 may be configured to removably couple to an arch 14. For example, as shown in FIGS. 16A and 16B, tension element 30 may include a coupler 90. Coupler 90 may be a hook, clasp, snap, screw, rail, latch, or any suitable mechanism for engage with an arch 14. When coupled to the arch 14, tension element 30 may be used to adjust the position of one or more arches 14. One or more portions of tension element 30 may be threaded to facilitate adjustment of the position of arch 14 when tension element adjustor 28 is actuated. Tension element 30 may therefore enable lower arch 18 to be adjusted anterior to upper arch 16 to move the users lower jaw in the anterior direction, which may help open the user's airway. In some embodiments, tension element 30 may be formed from a flexible material, such as, for example, nylon. This flexibility may allow the arch 14 that is coupled to tension element 30 to move slightly, which may allow some movement of the user's jaw and improve comfort.

FIG. 17A illustrates examples of lower arch 18 and tension element 30 that may be used in certain embodiments of adjustable breathing device 2. FIG. 17A depicts the tension element 30 of FIGS. 16A and 16B coupled with lower arch 18. In the illustrated embodiment, coupler 90 of tension element 30 is engaging with a corresponding portion of lower arch 18. This removable adjustment mechanism may allow the user to use arches 14 in other applications separate from adjustable breathing device 2. This removable adjustment mechanism may also improve the comfort and convenience of providing anterior jaw displacement by allowing the user to connect lower arch 18 to the adjustment mechanism (e.g., tension element 30 and tension element adjustor 28) if and when the user chooses.

FIG. 17B illustrates a portion of an example post 12 and FIG. 17C illustrates examples of an arched frame 50 and tension element 30 that may be used in certain embodiments of adjustable breathing device 2. The anterior portion of post 12 includes channels 24, tightening knob 26, countersink 36, slot 39, and tension element slot 40 (fastener 34 is not shown). In the embodiments shown, tension element slot 40 is a D-shaped slot and tension element 30 has a corresponding D-shaped cross-section. The corresponding D-shapes of tension element 30 and tension element slot 40 may substantially prevent tension element 30 from rotating when tension element adjustor 28 is used to adjust tension element 30. Although a D-shape is illustrated, other cross-sections may be used to prevent the rotation of tension element 30 in tension element slot 40. As one example, tension element slot 40 may have a “key” structure that corresponds with a channel in tension element 30.

FIG. 17D illustrates examples of nose mask 6, arches 14, and tension element 30 that may be used in certain embodiments of adjustable breathing device 2. As shown in FIG. 17D, in certain embodiments, at least a portion of tension element 30 may be threaded, and the threaded portion may engage with the tension element adjustor 28. Furthermore, tension element 30 may optionally pass through a portion of post 12. In some embodiments, tension element 30 may pass between flanges 22 and may pass above or under fastener 34. Adjusting tension element adjustor 28 may cause tension element to push or pull lower arch 18 in the anterior-posterior direction. Such adjustments may help to open the user's airway to improve breathing. While the illustrated embodiment does not depict mouth mask 8 or connector 10, other embodiments may include any suitable mouth mask 8 and connector 10.

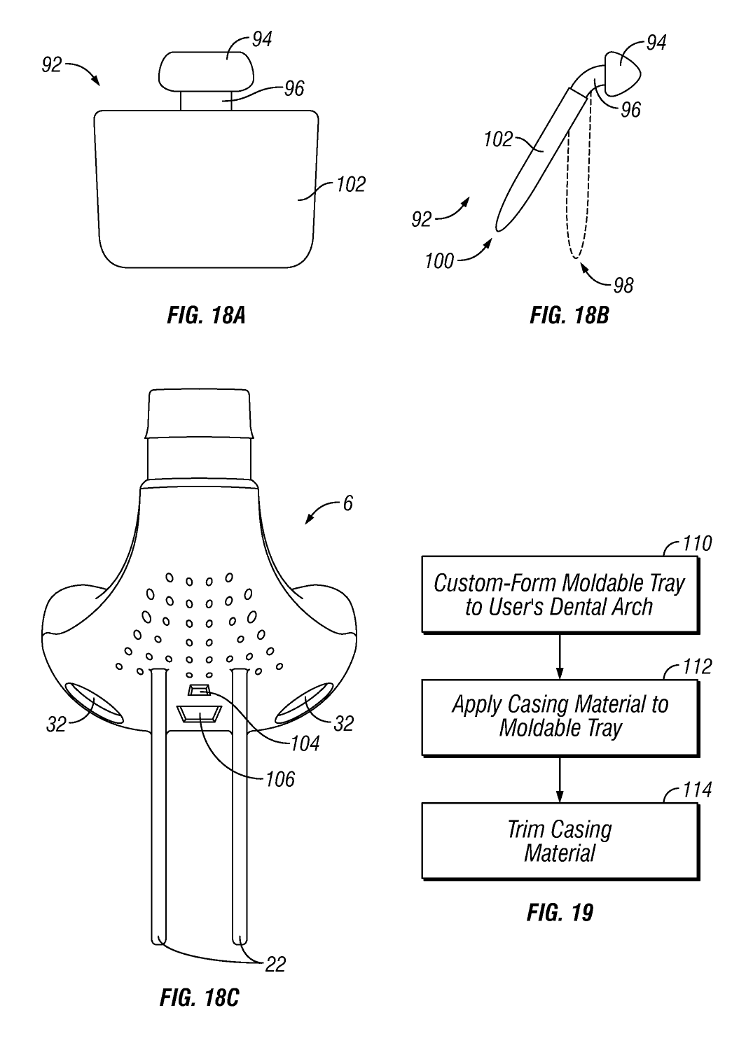

FIGS. 18A and 18B illustrate front and side views, respectively, of an example valve 92 that may be used in certain embodiments of adjustable breathing device 2. Valve 92 is configured to open in response to a change in pressure and may be included in certain embodiments of nose mask 6 and/or mouth mask 8. For example, valve 92 may be a “non-rebreathing” valve. Valve 92 may be configured to form a seal when the air pressure inside the mask is above a threshold value and to break the seal when the air pressure is below the threshold value. For example, valve 92 may be configured to break the seal when the internal pressure falls below 4 cm H₂O. Valve 92 may be composed of silicone, any other suitable material, or any combination thereof. Valve 92 may prevent the user from inhaling previously exhaled air if the gas delivery device is not providing air to the user. In the illustrated embodiment, valve 92 includes connector 94, hinge 96, and flap 102.

Connector 94 operates to connect valve 92 to adjustable breathing device 2. Connector 94 may be a hook, snap, plug, or any suitable component. Connector 94 may be composed of silicone, plastic, any suitable material, or any suitable combination thereof. In some embodiments, connector 94 may couple with an opening 104 (shown in FIG. 18B) or another attachment point of adjustable breathing device 2. In alternative embodiments in which valve 92 is integrally formed with adjustable breathing device 2, connector 94 may be omitted.

Hinge 92 allows valve 92 to move between closed position 98 and open position 100. Hinge 92 may be composed of silicone, plastic, any suitable material, or any suitable combination thereof. Hinge 92 may operate by bending, rotating, or any suitable movement. Closed position 98 may correspond to the position of valve 92 when adjustable breathing mask 2 is subjected to sufficiently high air pressure. Open position 98 may correspond to the position of valve 92 when adjustable breathing mask 2 is subjected to sufficiently low air pressure, which may prevent the user from inhaling previously exhaled air if the gas delivery device is not providing air to the user.

Flap 102 operates to seal an opening of adjustable breathing device 2 when valve 92 is in closed position 98. Flap 102 may be composed of silicone, plastic, any suitable material, or any suitable combination thereof. Flap 102 may form a seal when the air pressure inside adjustable breathing device 2 is above a threshold value, and it may break the seal when the air pressure is below the threshold value. In this manner, flap 102 may enable delivery of gas to the user from a gas delivery device, while also preventing the user from inhaling previously exhaled air if the gas delivery device is not providing air to the user.

FIG. 18C illustrates an example embodiment of nose mask 6 that may be used in certain embodiments of adjustable breathing device 2. Nose mask 6 includes flanges 22 and openings 32, 104, and 106. Openings 104 and 106 may also present in certain embodiments of mouth mask 8.

Opening 104 is configured to receive connector 94 such that opening 104 is substantially sealed when engaged by connector 94. In some embodiments, connect 94 may snap into or otherwise attach to opening 104. In other embodiments, opening 104 may not be present, and connector 94 may couple with nose mask 6 via a clasp, hook, or other attachment point.

Opening 106 is configured to receive flap 102 such that opening 106 is substantially sealed when engaged by flap 102. Opening 106 may be any suitable shape and/or size to accommodate flap 102. When flap 102 is not sealing opening 106, gas may pass out of the interior space of nose mask 6 via opening 106. Flap 102 may disengage from opening 106 in response to a drop in the internal air pressure of nose mask 6 to prevent the user from inhaling previously exhaled air if the gas delivery device is not providing air to the user.

FIG. 19 illustrates an example method of forming a moldable tray at least partially covered by a casing in accordance with certain embodiments of the present disclosure. The method is provided merely as an example, and other embodiments may utilize some, all, or none of these steps.

At step 110, a moldable tray is custom-formed to a user's dental arch. Certain embodiments may use any type or configuration of moldable tray 70 discussed above. Furthermore, any suitable method of custom-forming the moldable tray to the user's dental arch may be used. For example, in some embodiments, a cast of the user's dental arch is constructed, and the moldable tray is pressed against the cast to form an impression of the user's dental arch. In such embodiments, a cast can be formed using any suitable method. For example, after taking an impression of the user's dentition, a cast can be constructed based on this impression. Such casts can be made from any suitable material, including, for example, stone, plastic, or any material that is suitably rigid to allow for the custom-forming of a moldable tray against the cast. In other embodiments, a moldable tray is pressed against the user's dentition to form an impression of the user's dental arch. Such embodiments may allow custom-formed moldable trays to be produced without the additional expense of creating a cast of the user's dentition.

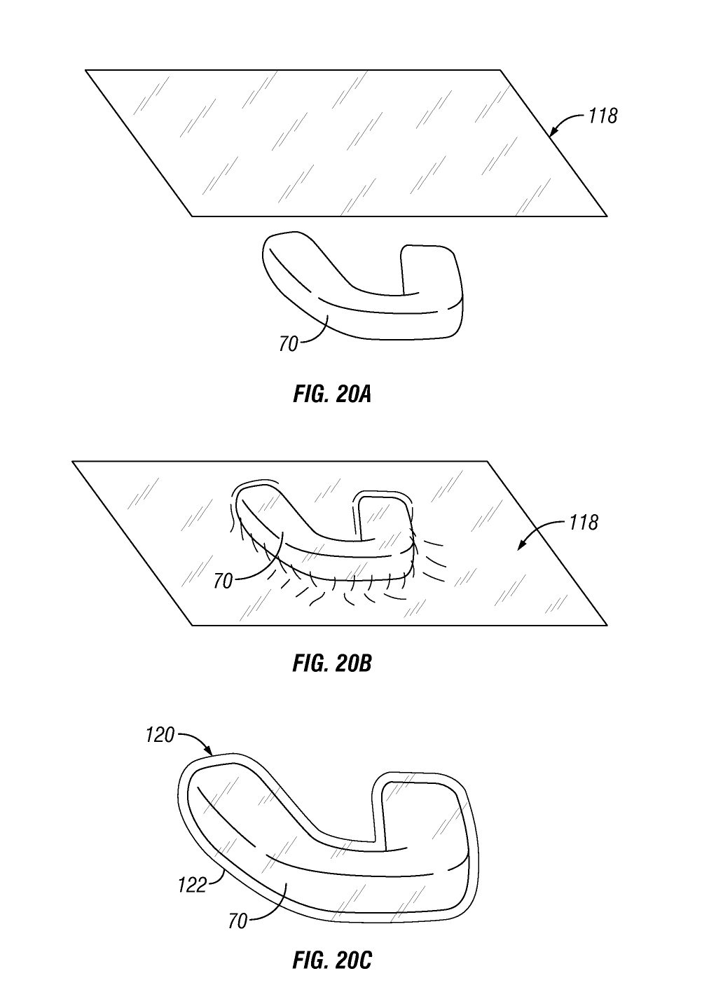

At step 112, a casing material is applied to the moldable tray. In some embodiments, the casing material is applied by applying pressure to blow down the casing material around a portion of the moldable tray. For example, positive pressure may be applied from above, negative pressure may be applied from below (e.g. by creating at least a partial vacuum), or any other suitable method of applying the casing material to the moldable tray by creating a pressure differential may be used. Other embodiments may use any suitable method of applying a casing material to a portion of the moldable tray. In certain embodiments, the casing material may be heated before being applied to the moldable tray in order to render the casing material sufficiently malleable to conform to a portion of the moldable tray. Furthermore, in certain embodiments, an adhesive may be applied to allow the casing material to adhere to the moldable tray, while in other embodiments, the casing material may adhere to the moldable tray due to the physical properties of the casing material or moldable tray. The casing material may be plastic, polycarbonate, nylon, acrylic, or any suitable material. The casing material are discussed further below with respect to casing material 118 of FIG. 20A.

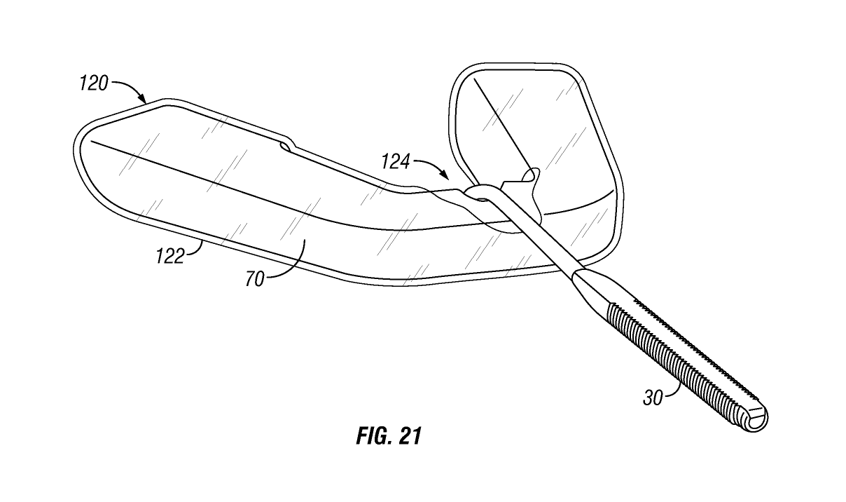

At step 114, the casing material is trimmed. In some embodiments, excess material may be trimmed or otherwise removed from the moldable tray. For example, in embodiments in which a plastic sheet is blown down over the moldable tray, excess material may extend beyond the edge of the moldable tray following the blow down process (as shown, for example, in FIG. 20B). Such material can be trimmed so that the casing does not extend substantially beyond the edge of the moldable tray. An example of the resulting configuration of the casing material and moldable tray is shown in FIGS. 20C and 21.

Forming an encased moldable tray as described above may improve the durability of the moldable tray, which can reduce the need for repairs or replacement. Furthermore, forming the casing as described above may reduce manufacturing costs. For example, applying the casing material as described above may use less expensive materials or may provide a simplified or less expensive technique for forming the casing. Moreover, using a substantially rigid but sufficiently flexible casing material may also reduce the need for precise configuration or alignment of components. For example, such techniques may result in a moldable tray (or associated components) having an improved range of motion, which may mitigate problems caused by the slight misalignment of certain components or attachments.

Various embodiments may perform some, all, or none of the steps described above. For example, some embodiments may use a casing material that is shaped such that excess casing material need not be trimmed, in which case step 114 may be omitted. Furthermore, certain embodiments may perform additional steps. For example, the moldable tray may be heated before it is custom-formed to the user's dental arch. As another example, lining materials (such as, for example, THERMACRYL or other suitable materials) may be applied to one or more portions of moldable tray 70 before or after the application of casing material 118. In such embodiments, additional lining materials may also be applied at a later date if the original lining becomes worn. The above steps may be used in conjunction with any suitable materials, techniques, or devices described herein.

FIGS. 20A-20C illustrate example steps during an example process of forming an encased moldable tray. In the illustrated embodiments, casing 120 is formed by applying casing material 118 to moldable tray 170 and then trimming or otherwise removing excess material.

FIG. 20A illustrates casing material 118 positioned above moldable tray 70. Casing material 118 may be any material that is capable of being formed around moldable tray 70. For example, casing material 118 can be plastic (such as, for example, polycarbonate), nylon, acrylic, or any other suitable material. In the illustrated embodiment, casing material 118 is initially formed as a generic sheet. Such embodiments may provide a cost-effective method of forming an encased, custom-formed moldable tray from non-custom components. In other embodiments, casing material 118 may be pre-formed to approximately match the shape of moldable tray 70, which may reduce or eliminate the need for trimming after casing material 118 is applied.

FIG. 20B illustrates an example intermediate product after applying casing material 118 to moldable tray 70. In the illustrated embodiment, casing material 118 is shown blown down against moldable tray 70. This configuration may be created by the process described above with respect to step 112 of FIG. 19. Casing material 118 may not be applied to the portion of moldable tray 70 that is custom-formed to the user's dentition. For example, as shown in FIG. 20B, casing material 118 substantially surrounds the top and lateral surfaces of moldable tray 70 but not the bottom of moldable tray 70, though this is not required.

FIG. 20C illustrates moldable tray 70 partially surrounded by casing 120. Casing 120 may be formed from any material discussed above with respect to casing material 118. In some embodiments, casing 120 may be formed by trimming or otherwise removing excess portions of casing material 118 at edge 122 after casing material 118 is applied to moldable tray 70. In some embodiments, casing 120 has a thickness ranging from 0.5-2 millimeters, though other embodiments may have any suitable thickness. Casing 120 may substantially surround the lateral surfaces of moldable tray 70 and the surface of moldable tray 70 that is opposite the impression of the occlusal surface of the user's dentition. For example, in the illustrated embodiment, an impression of the user's dentition (not shown) is formed on the underside of moldable tray 70, and casing 120 substantially surrounds the surface opposite the impression of the user's dentition (the upper surface as shown in FIG. 20C) as well as the buccal and lingual sides of moldable tray 70. In other embodiments, casing 120 is formed on the surface of moldable tray 70 opposite the impression of the user's dentition and on one or more portions of the buccal or lingual surfaces of moldable tray 70. In still other embodiments, casing 120 may be formed only on the surface of moldable tray 70 opposite the impression of the occlusal surface of the user's dentition. In certain embodiments, casing 120 is not applied to, or is removed from, the portion of moldable tray 70 that has been custom-formed to the user's dentition. Casing 120 may improve the durability of moldable tray 70, providing improved resistance to wear and tear. Improved durability may reduce the need to repair or replace moldable tray 70. Furthermore, casing 120 may improve the ability of moldable tray 70 to distribute force from or otherwise mitigate bruxing.

FIG. 21 illustrates an example moldable tray and an example casing. In the illustrated embodiment, tension element 30 is connected to moldable tray 70, which is partially surrounded by casing 120. Casing 120, which includes opening 124, extends around the upper portion of moldable tray 70 down to edge 122.

Opening 124 may be any opening in casing 120 that is capable of admitting an attachment to moldable tray 70. In the illustrated embodiment, opening 124 allows tension element 30 to pass through casing 120 and connect to moldable tray 70. Such connections may be removable or permanent. Opening 124 may have any suitable configuration to admit a dental attachment, and casing 120 may include any suitable number of openings 124. In some embodiments, opening 124 is formed in casing material 118 before casing material 118 is applied to moldable tray 70. In other embodiments, opening 124 is created after applying casing material 118 to moldable tray 70. For example, in some embodiments, after blowing down casing material 118 over moldable tray 70, a portion of casing material 118 may be cut out or otherwise removed to form opening 124. In such embodiments, casing material 118 may be applied while a dental attachment is connected to moldable tray 70, and opening 124 may be cut out around the attachment. Furthermore, the dental attachment may have a protective shielding to prevent casing material 118 from sticking to the attachment or to protect the attachment during the cutting of opening 124. For example, a portion of tension element 30 may be covered by shielding (such as, for example, wax, plastic, metal, or any other suitable material) to protect it while opening 124 is formed. Opening 124 may allow breathing devices to obtain the benefits of casing 120 while still allowing for the connection of various attachments.

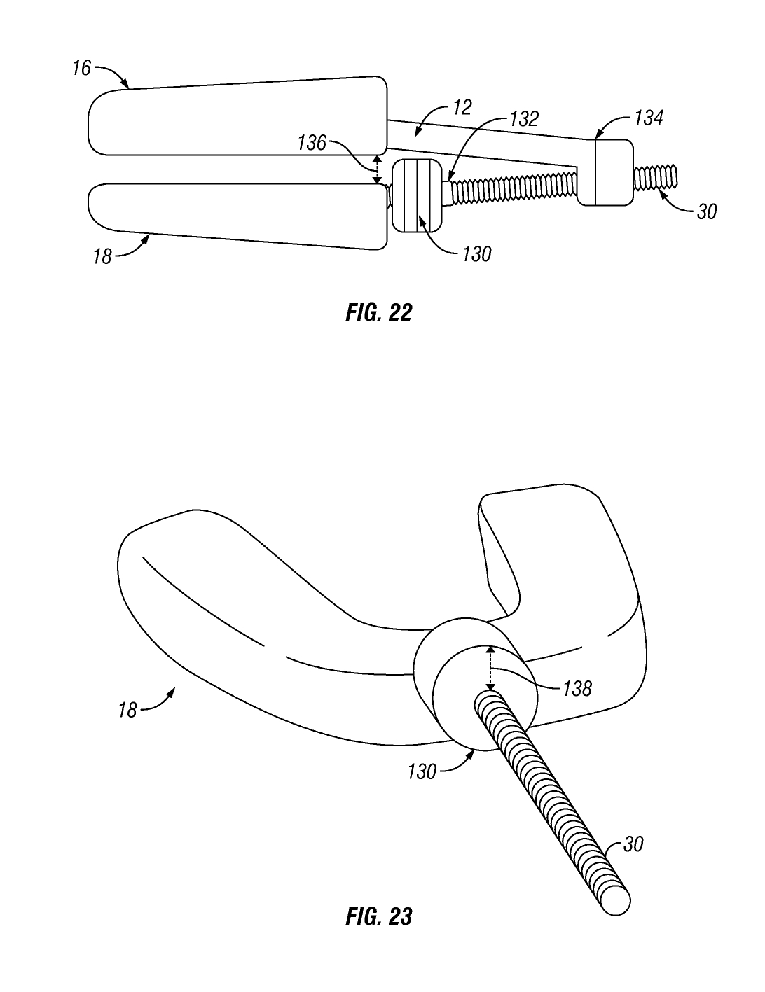

FIG. 22 illustrates a side view of an example breathing device using an example separator. In the illustrated embodiment, the breathing device includes post 12, upper arch 16, lower arch 18, tension element 30, and separator 130. FIG. 23 illustrates an isometric view of lower arch 18, tension element 30, and separator 130.

Separator 130 may be any component capable increasing the separation between upper arch 16 and lower arch 18. Separator 130 can be formed from plastic (such as, for example, polycarbonate), rubber, nylon, silicone, SANTOPRENE, any other suitable material, or any combination thereof. Separator 130 may be sufficiently rigid to provide a separation between upper arch 16 and lower arch 18 when the user is not applying substantial biting force. Furthermore, in some embodiments, separator 130 may be sufficiently flexible to provide limited elasticity when the user applies biting force, which may improve comfort. More rigid embodiments may provide more definite spacing between the arches. In the illustrated embodiment, separator 130 is a ring that is configured to fit around tension element 30, though other embodiments may use any suitable component. In some embodiments, separator 130 may be slide down tension element 30 and into place adjacent to or near the arches. In other embodiments, separator 130 may be snapped, clipped, or otherwise attached without sliding down tension element 30. Furthermore, separator 130 may be attached to any suitable component. For example, while shown in FIG. 22 as connected to tension element 30, in other embodiments, separator 130 may be attached to post 12, upper arch 16, lower arch 18, or any suitable component.

Distance 132 represents the distance between upper arch 16 and lower arch 18 created by separator 130. When separator 130 is in its operative position (as shown, for example, in FIG. 22), separator 130 may abut post 12 or otherwise cause upper arch 16 and lower arch 18 to be separated by distance 132. Width 134 represents the width of separator 130. In some embodiments, width 134 is between 2 and 10 millimeters, though these dimensions are not required. As shown in FIGS. 22 and 23, increasing width 134 operates to increase distance 132. In some embodiments, a kit is provided that includes multiple separators 130. In such embodiments, each separator 130 may have a different width 134, which can be printed on its side, though this is not required. For example, three separators 130 may be provided having respective widths 134 of 2 mm, 4 mm, and 6 mm, though other embodiments may have different numbers or sizes of separators 130. In other embodiments, separator 130 may have a non-uniform cross-section. For example, separator 130 may be oblong such that the operative width (i.e. the width of the portion of separator 130 that effects the separation of the arches by distance 132) is different depending on the orientation of separator 130. Such embodiments may mitigate the need for multiple separators 130 of different sizes, since a single separator 130 may be adjusted to provide different amounts of separation. By increasing the separation of the arches, separator 130 may increase the open area in the user's oral cavity, which may improve breathing.

Ridge 136 may be any structure that is capable of providing an adjustment point for separator 130. In some embodiments, tension element 30 may include ridge 136 or another feature that provides a distinct adjustment point for separator 130. For example, in the illustrated embodiment, separator 130 may slide over ridge 136 so that it is secured in its operative position. Some embodiments may have multiple ridges 136. For example, tension element 30 or post 12 may have multiple ridges 136 to provide different adjustment points for separator 130. In embodiments where post 12 is connected to tension element 30 at an angle, more posterior adjustment points may provide greater amounts of separation between the arches by increasing the angle between post 12 and tension element 30. Such embodiments may provide a more predictable mechanism for creating different distances 132 with a single separator 130.

Point 138 represents a portion of post 12 that is capable of accommodating different angles of connection to adjacent components. In some embodiments, by separating upper arch 16 and lower arch 18, separator 130 may change the angle at which post 12 meets the components to which it is connected. For example, in the illustrated embodiment, separator 130 may change the angle at which post 12 meets upper arch 16 and tension element 30. In such embodiments, post 12 or its connected components may be flexible or may otherwise accommodate the change in this angle. For example, point 138 (or another portion of post 12 or upper arch 16) may be flexible, hinged, or otherwise configured to accommodate different angles of connection.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure encompasses all changes, substitutions, variations, alterations, and modifications to the example embodiments herein that a person having ordinary skill in the art would comprehend. For example, while the embodiments of FIGS. 1-17 are shown as having particular connectors 10, nasal pillows 20, flanges 22, and channels 24, any suitable number, type, and configuration of these components may be used. As another example, any suitable type and configuration of nose mask 6 and mouth mask 8 may be used. As yet another example, nose mask 6, mouth mask 8, and arches 14 may be integrally formed or adjustably coupled using any suitable type of connection. Furthermore, post 12 may use any suitable mechanism to adjustably secure nose mask 6 and mouth mask 8. Additionally, certain embodiments may use any suitable number, type, and configuration of separators 130 or ridges 132.

Moreover, although this disclosure describes and illustrates respective embodiments herein as including particular components, elements, functions, operations, or steps, any of these embodiments may include any combination or permutation of any of the components, elements, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative.

What is claimed is:

1. A breathing device comprising:
a first mask configured to deliver gas to a user's nose when the first mask is connected to a gas delivery device, the first mask defining a first interior space;
a second mask configured to form a substantial seal around the user's oral cavity when pressed against a portion of the user's mouth, the second mask defining a second interior space;
an oral appliance arch; and
a post coupled to the oral appliance arch and configured to adjustably secure the first mask relative to the oral appliance arch;
wherein the second mask is configured to be adjustably secured to the post, and the first and second masks are separately adjustable relative to the oral appliance arch.

2. The breathing device of claim 1, wherein the second mask is configured to be adjustable in the anterior-posterior direction relative to the user.

3. The breathing device of claim 2, wherein the second mask comprises a sealing portion defining an opening, the sealing portion configured to form a substantial seal around the post.

4. The breathing device of claim 3, wherein the sealing portion is further configured to substantially secure the second mask relative to the post.

5. The breathing device of claim 4, wherein the second mask is substantially secured relative to the post by frictional force between the sealing portion and the post.

6. The breathing device of claim 3, wherein the post comprises a plurality of notches defining discrete adjustment points for the second mask along the post.

7. The breathing device of claim 1, further comprising a connector defining a third interior space, the connector configured to connect the first and second masks, wherein the third interior space is contiguous with the first and second interior spaces when the connector is connecting the first and second masks.

8. The breathing device of claim 7, wherein the connector comprises a flexible tube.

9. The breathing device of claim 1, wherein the oral appliance arch comprises a thermoplastic material cross-linked by radiation.

10. A kit for constructing an adjustable breathing device, the kit comprising:
a first mask configured to deliver gas to a user's nose when the first mask is connected to a gas delivery device, the first mask defining a first interior space;
a second mask configured to form a substantial seal around the user's oral cavity when pressed against a portion of the user's mouth, the second mask defining a second interior space;
a post configured to adjustably secure the first mask relative to the post; and
a plurality of nasal pillows, each nasal pillow comprising:
a first end defining a circumference and configured to be positioned proximate to the user's nostril; and
a second end configured to couple to the first mask;
wherein the second mask is configured to be adjustably secured to the post, and the first and second masks are separately adjustable relative to the post.

11. The kit of claim 10, further comprising an oral appliance arch configured to couple to the post.

12. The kit of claim 10, wherein the plurality of nasal pillows comprises first and second nasal pillow pairs, the circumferences of the first ends of the nasal pillows of the first nasal pillow pair being different from the circumferences of the first ends of the nasal pillows of the second nasal pillow pair.

13. The kit of claim 10, wherein the plurality of nasal pillows comprises first and second nasal pillow pairs, each nasal pillow of the first nasal pillow pair extending a first distance from the nose mask when attached to the nose mask, each nasal pillow of the second nasal pillow pair extending a second distance from the nose mask when attached to the nose mask, the first distance being different from the second distance.

14. The kit of claim 10, wherein the second mask is configured to be adjustable in the anterior-posterior direction along the post.

15. The kit of claim 10, wherein the second mask comprises a sealing portion defining an opening, the sealing portion configured to form a substantial seal around the post.

16. The kit of claim 10, wherein the sealing portion is further configured to substantially secure the second mask relative to the post.

17. The kit of claim 10, wherein the second mask is substantially secured relative to the post by frictional force between the sealing portion and the post.

18. The kit of claim 10, wherein the post comprises a plurality of notches configured to engage the sealing portion of the second mask.

19. The kit of claim 10, further comprising a connector defining a third interior space, the connector configured to connect the first and second masks, wherein the third interior space is contiguous with the first and second interior spaces when the connector is connecting the first and second masks.

20. The kit of claim 11, wherein the oral appliance arch comprises a thermoplastic material cross-linked by radiation.

21. A breathing device comprising:
a first mask configured to deliver gas to a user's nose when the first mask is connected to a gas delivery device;
a first flange connected to the first mask;
a second mask configured to form a substantial seal around the user's oral cavity when pressed against a portion of the user's mouth;
a first oral appliance arch; and
a post coupled to the first oral appliance arch and configured to adjustably secure the first mask relative to the first oral appliance arch, the post comprising:
a second flange;
a fastener configured to adjustably secure the first flange against the second flange;
wherein the second mask is configured to be adjustably secured to the post and to be adjustable in the anterior-posterior direction along the post, and the first and second masks are separately adjustable relative to the first oral appliance arch.

22. The breathing device of claim 21, wherein:
the first oral appliance arch comprises:
arched frame comprising polycarbonate; and
an arched moldable tray in communication with the arched frame, the arched moldable tray comprising polycaprolactone.

23. The breathing device of claim 21, wherein the post further comprises a third flange, the second and third flanges defining a channel configured to receive the first flange.

24. The breathing device of claim 21, further comprising:
a second oral appliance arch; and
a tension element configured to couple with the second oral appliance arch and adjust a position of the second oral appliance arch in the anterior-posterior direction relative to the first oral appliance arch.

25. The breathing device of claim 24, wherein the tension element comprises nylon.

26. The breathing device of claim 21, wherein the first oral appliance arch comprises a thermoplastic material cross-linked by radiation.

27. A method of forming a breathing device, the method comprising:
custom-forming an oral appliance arch to a user's dentition, the custom-formed oral appliance arch configured to be positioned proximate to an occlusal surface of a user's dental arch such that the custom-formed oral appliance arch extends beyond the cuspids of the user's dental arch when the custom-formed oral appliance arch is inserted into the user's mouth, the custom-formed oral appliance arch comprising:
a first surface having an impression of an occlusal surface of the user's dentition;
a second surface opposite the first surface; and
applying a casing material at least to the second surface of the oral appliance arch.

28. The method of claim 27, wherein:
the custom-formed oral appliance arch further comprises:
a buccal surface configured to be positioned proximate to the user's cheek when the custom-formed oral appliance arch is inserted into the user's mouth; and
a lingual surface configured to be positioned proximate to the user's tongue when the custom-formed oral appliance arch is inserted into the user's mouth; and
applying the casing material comprises applying the casing material to the second surface, the buccal surface, and the lingual surface of oral appliance arch.

29. The method of claim 28, wherein applying the casing material comprises applying the casing material to the oral appliance arch such that the casing material substantially conforms to the second surface, the buccal surface, and the lingual surface of the oral appliance arch.

30. The method of claim 27, wherein applying the casing material comprises creating a pressure differential between opposite sides of the casing material.

31. The method of claim 27, further comprising trimming the casing material substantially proximate to an edge of the buccal surface of the oral appliance arch.

32. The method of claim 27, wherein in the casing material comprises one or more of the following materials:
polycarbonate;
nylon; and
acrylic.

33. A breathing device comprising:
a custom-formed oral appliance arch configured to be positioned proximate to an occlusal surface of a user's dental arch such that the custom-formed oral appliance arch extends beyond the cuspids of the user's dental arch when the custom-formed oral appliance arch is inserted in the user's mouth, the custom-formed oral appliance arch comprising:
a first surface having an impression of an occlusal surface of the user's dental arch; and
a second surface opposite the first surface; and
a casing coupled to the custom-formed oral appliance arch, the casing substantially conforming at least to the second surface of the custom-formed oral appliance arch.

34. The breathing device of claim 33, wherein:
the custom-formed oral appliance arch further comprises:
a buccal surface configured to be positioned proximate to the user's cheek when the custom-formed oral appliance arch is inserted into the user's mouth; and
a lingual surface configured to be positioned proximate to the user's tongue when the custom-formed oral appliance arch is inserted into the user's mouth; and
the casing material is further coupled to the buccal and lingual surfaces of the oral appliance arch.

35. The breathing device of claim 34, wherein the casing material substantially conforms to the second surface, the buccal surface, and the lingual surface of the oral appliance arch.

36. The breathing device of claim 33, wherein the casing comprises an outer edge that substantially aligns with an edge of the buccal surface of the oral appliance arch.

37. The breathing device of claim 33, wherein in the casing comprises one or more of the following materials:
polycarbonate;
nylon; and
acrylic.

38. A kit for constructing an adjustable breathing device, the kit comprising:
a first oral appliance arch;
a second oral appliance arch;
a post coupled to the first oral appliance arch;
a tension element configured to couple with the second oral appliance arch and adjust a position of the second oral appliance arch in the anterior-posterior direction relative to the first oral appliance arch; and
one or more separators configured to separate the first and second oral appliance arches when the first and second oral appliance arches or positioned proximate to a user's dentition.

39. The kit of claim 38, wherein the one or more separators comprises a plurality of separators, each separator having a different respective width.

40. The kit of claim 38, wherein each of the one or more separators has a width between 2 and 10 millimeters.

41. The kit of claim 38, wherein each of the one or more separators defines an opening configured to receive the tension element.

42. The kit of claim 38, wherein the tension element comprises one or more ridges, each ridge providing a distinct adjustment point for one of the one or more separators.

43. The kit of claim 40, wherein each of the one or more separators comprises a ring.
